(19) United States
(12) Patent Application Publication
(10) Pub. No.: US 2005/0112701 A1
(43) Pub. Date: May 26, 2005

(54) Test system for the identification of APJ receptor ligands

(75) Inventors: Petra Arndt, Bickenbach (DE); Daniel Margerie, Frankfurt am Main (DE); Goetz Muench, Munich (DE); Martin Ungerer, Munich (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt (DE)

(21) Appl. No.: 10/931,080

(22) Filed: Aug. 31, 2004

Related U.S. Application Data
(60) Provisional application No. 60/538,648, filed on Jan. 23, 2004.

(30) Foreign Application Priority Data
Sep. 11, 2003 (EP) 03020430.9

(51) Int. Cl. G01N 33/53; G01N 33/567; C12N 15/85

(52) U.S. Cl. 435/7.2; 435/455

(57) ABSTRACT

The present invention refers to a test system for the identification of a ligand for angiotension receptor like-1 (APJ receptor) comprising an APJ receptor or a functional variant thereof and a Gαq or a Gαl protein or a hybrid protein which is assembled from portions of different G proteins like Gαq or Gαl proteins, a method of screening an APJ receptor ligand using the test system, a method for producing a medicament, a method for diagnosis and prognosis of a heart disease and a kit usable for detection or prognosis of a heart disease.

The present invention refers to a test system for the identification of a ligand for angiotension receptor like-1 (APJ receptor) comprising an APJ receptor or a functional variant thereof and a Gαq or a Gαl protein or a hybrid protein which is assembled from portions of different G proteins like Gαq or Gαl proteins, a method of screening an APJ receptor ligand using the test system, a method for producing a medicament, a method for diagnosis and prognosis of a heart disease and a kit usable for detection or prognosis of a heart disease.

Within a multicellular organism cells have to communicate with each other to ensure the existence of the whole organism. Due to specialization of each cell, the characteristic function of each cell contributes to the structure and activity of the whole organism. These functions are in general under the control of extracellular signals, which include hormones, neurotransmitters, local hormone etc. Almost all the extracellular signals consist of a change in concentration of a given type of molecule in the environment of the cell. In general the extracellular signal has to be translated into an intracellular signal.

There are different mechanisms by which extracellular signals are received by a cell. Many signal substances are not able to penetrate through the cellular membrane and enter into the cell. Therefore, these extracellular signals have to be transduced into an intracellular signal. A quite successful system for the signal transduction consists of three components, i.e. a G protein coupled receptor, a G protein and an effector protein.

G Protein Coupled Receptors

The family of G protein coupled receptors is a large superfamily of integral membrane proteins. Currently there are about 2,000 G protein coupled receptor sequences available from public databases. Some of the receptors and their endogenous ligands as well as exogenous agonists and antagonists have been known for a long time (e.g. the β₂-adrenergic receptor). Others are so-called orphan G protein coupled receptors since they do not bind to any known endogenous ligand.

G protein coupled receptors (also known as 7-transmembrane, heptahelical or serpentine receptors) exhibit amino acid sequence as well as structural similarities. They are composed of a single polypeptide chain which spans the cell membrane seven times and consist of an extracellular amino terminus, seven predominantly hydrophobic α-helical domains (of about 20-30 amino acids) spanning the cell membrane, approximately 20 well-conserved amino acids and a cytoplasmic carboxy terminus.

The binding of an agonist to its receptor mediates a change in receptor conformation whereby the receptor is transferred to its active form. The receptor in its active form activates the G protein and mediates the exchange of GTP for GDP. Antagonists (blockers) stabilise the receptor in its inactive form thus blocking the prevailing downstream signal transduction pathway.

G Proteins

G protein coupled receptors transduce the signals via heterotrimeric guanine nucleotide-binding proteins (G proteins). Heterotrimeric G proteins consist of an α (39-42 kDa), a β (35-36 kDa), and a γ subunit (7-10 kDa). The α subunit harbours a binding site for GTP/GDP. G proteins are naturally occurring on the cytoplasmic side of the plasma membrane.

Binding of an extracellular ligand leads to a conformational change in the receptor protein that allows it to make contact with a guanine-nucleotide binding protein (G protein). The activated G protein coupled receptor alters the confirmation of the α subunit and enhances the exchange of GTP for GDP. In the inactive state the βγ dimer is bound to the a subunit. Upon the exchange, the βγ dimer dissociates from the α subunit. Both the activated a subunit and the βγ dimer can influence intracellular effector proteins. The α subunit then catalyses the hydrolysis of GTP into GDP and Pᵢ, which causes the inactivation of the α subunit and subsequently the binding of the βγ dimer to the α subunit.

Presently, the G protein α subunit family is grouped in four different classes, i.e. G_{αs}, G_{αi}, G_{αq} and G_{α12}. G_{αs} and G_{αi} classes stimulate or inhibit adenylyl cyclase activity, respectively. The G_{αq} class stimulates phospholipase Cβ and includes G_{αq}, G_{α11}, G_{α14} and G_{α15/16}. G_{α12} is a class similar to, yet distinct from, G_{αq}.

In general, G protein coupled receptors activate specifically a particular G protein α subunit family, which leads to the activation or inactivation of a particular signal transduction pathway.

Effector Proteins

G protein effect a wide spectrum of biological activities through various intracellular enzymes, ion channels and transporters.

The activated G protein activates or inactivates various signal pathways, thereby activating or inactivating further proteins such as enzymes and ion channels which are called effector proteins. Typical effector proteins are ion channels such as N-type Ca²⁺ channel and G protein coupled inwardly rectifying K⁺ channel (GIRK) and enzymes such as adenylyl cyclase and phospholipase C.

APJ Receptor

One of the earliest orphan receptors was the APJ receptor (angiotensin receptor like-1), originally identified by O'Dowd et al. (O'Dowd et al., 1993, Gene 136: 355-360). Recently, the endogenous ligand of the APJ receptor, apelin-36, has been identified (Tatemoto et al., 1998, Biochem Biophys Res Commun 251: 471-476). Apelin-36 is a peptide agonist consisting of 36 amino acids, but also shorter variants of apelin-36 (C-terminal peptides of 13-19 amino acids) exhibited agonist properties on the APJ receptor. Apelin is produced through processing from the C-terminal portion of the pre-proprotein consisting of 77 amino acid residues and exists in multiple molecular forms.

Apelin and APJ receptor mRNA have been found to be ubiquitously expressed in peripheral tissue as well as in various regions of central nervous system and also in particular peripheral blood mononuclear cells. In the immune system the APJ receptor is reported to support the entry HIV-1 as a co-receptor with CD-4 (Cayabyab et al., 2000, J Virol 74: 11972-11976). Additionally, it was recently found that the APJ receptor exhibits high level of mRNA expression in the heart (Lee et al., 2000, J Neurochem 74: 34-41). Activity of the endogenous ligand was found in extracts of bovine brain, intestine stomach tissue as well as in the heart.

Apelin has shown to be involved in the suppression of cytokine production from mouse spleen, the promotion of extracellular acidification and inhibition of cAMP production in Chinese hamster ovary cells and in the regulation of blood pressure and blood flow (De Falco at al, 2002, In Vivo 2002 16: 333-336). In the heart it has been shown that apelin induced a dose-dependent positive inotropic effect which seems to be mediated via phospholipase C and protein kinase C (Szokodi et al., 2002, Circ Res 91: 434-440). Furthermore, selective inhibitors of Na⁺/H⁺ exchange isoform-1 (NHE) and reverse mode Na⁺/Ca²⁺ exchange (NCX) significantly suppressed response of apelin, indicating their involvement in the signal transduction pathway (Szokodi et al., supra).

Surprisingly, it was now found that cardiovascular diseases are associated with increased expression of the APJ receptor gene resulting in increased level of APJ receptor mRNA as well as APJ receptor protein, which is contrary to the findings in WO 03/013576. Furthermore, apelin expression is enhanced, too. Heart diseases being associated with the increased expression are in particular congestive dilated cardiomyopathy, hypertrophic cardiomyopathy and coronary artery disease.

Coronary Artery Disease

Coronary artery disease is the most common form of heart disease. The symptoms include angina pectoris, myocardial infarction, shortness of breath, irregular or faster heart beats etc. It most often results from atherosclerosis, which is caused by plaques made of cholesterol, fatty compounds, calcium, and fibrin. Atherosclerosis of the coronary arteries leads to a reduced lumen of these vessels. As the lumen narrows, resistance to flow increases and myocardial blood flow is compromised and an adequate supply with oxygen is no more guaranteed. In addition to thrombus formation initiated by platelet aggregation, the myocardium may also be injured due to coronary artery disease by one of the other mechanisms. The therapy for coronary artery disease is focused on assuring the sufficient blood flow to the heart and includes medication (β blockers, calcium channel blockers, aspirin, statins and nitrates), angioplasty and bypass surgery.

Cardiomyopathy

This disease of the heart muscle, which is not caused by coronary atherosclerosis, leads to an increased peripheral resistance in lesser or greater circulation. In some instances, heart rhythm is disturbed, leading to irregular heartbeats, or arrhythmias. Usually, the exact cause of the muscle damage is never found, but it is a leading reason for heart transplantation. There are four types of cardiomyopathy: nonischemic cardiomyopathy, congestive dilated cardiomyopathy, hypertrophic cardiomyopathy and restrictive cardiomyopathy.

Congestive Dilated Cardiomyopathy

This disease is associated with an enlargement of at least one ventricle which causes a reduction of the heart's pumping ability. As a consequence of this the heart tries to cope with the pumping limitation by further enlarging and stretching (compensation). Patients suffering from congestive dilated cardiomyopathy show in general fatigue, weakness, shortness of breath and swelling of the legs and feet, resulting from fluid accumulation that may also affect the lungs (congestion) and other parts of the body. In most cases a specific cause for the disease is never identified. But some factors such as alcohol, viral infections and particular drugs have been linked to the disease's occurrence. The disease is treated by avoiding complicating factors and controlling the symptoms. Therapy begins with the elimination of obvious risk factors, such as alcohol consumption. Additionally, weight loss and dietary changes may be helpful.

Hypertrophic Cardiomyopathy

In hypertrophic cardiomyopathy, the walls of the left ventricle become thickened and stiff. The greatest thickening tends to occur in septum. The thickening reduces the flow of blood through the heart. Most cases of hypertrophic cardiomyopathy are inherited. In other cases, there is no clear cause. If patients suffering from hypertrophic cardiomyopathy show symptoms (many do not) they suffer from breathlessness and chest discomfort. Other signs are fainting during physical activity, strong rapid heartbeats and fatigue. In advanced stages of the disease, patients may have severe heart failure and its associated symptoms, including fluid accumulation or congestion. Treatments for hypertrophic cardiomyopathy vary but can include reduced physical activity, drugs (β blockers, calcium channel blockers, antiarrhythmic medications and diuretics), pacemakers and surgery.

The fact that APJ receptor and apelin are associated with heart diseases makes them an interesting pharmacological target. This applies particularly with regard to the difficulties in treating heart diseases as detailed above. In many cases a successful and/or long-term therapy or even prophylaxes does not existent.

One object of the present invention was therefore the development of a test system for the identification and characterisation of APJ receptor ligands, which may be used e.g. in the prevention or treatment of heart diseases.

Surprisingly it was found that a test system for the identification of an APJ receptor ligand comprising
(a) an APJ receptor or a functional variant thereof, and
(b) a Gαq or a Gαi protein or a hybrid protein which is assembled from portions of different G proteins like Gαq or Gαi proteins (e.g. a G_{αΔ6qi4myr} protein),
   wherein the binding of said APJ receptor ligand to said APJ receptor or functional variant thereof effects a measurable or detectable signal.

An APJ receptor ligand is any compound which binds specifically to the APJ receptor. A specific binding to the APJ receptor according to the present invention includes, without limitation, binding with a dissociation constant K_{D} of not exceeding 10⁻⁴ mol/l, preferably not exceeding 10⁻⁵ mol/l. The dissociation constant K_{D} can determined in competition binding experiments using membranes of human ventricle and [¹²⁵I]-Pyr¹)Apelin-13 as radioligand (Katugampola et al, 2001, Br J Pharmacol 132:1255-1260) according to the following equation:
B[L]=[L]/{[L]+K_{DL}(1+[L*]/K_{DL*}},
wherein [L] and [L*] represent the concentration of the APJ receptor ligand and the radioligand, respectively. K_{DL} and K_{DL*} are the dissociation constants of the APJ receptor ligand and the radioligand, respectively and B[L] is the binding (%) at a particular concentration of the APJ receptor ligand.

In an preferred embodiment of the invention the ligand to be identified is an agonist or an antagonist. Agonists bind to the APJ receptor, induce a conformational change of the same and activate the respective signal transduction pathway which leads to a measurable or detectable signal. Antagonists or blockers also bind to the receptor but do in general not induce signal transduction.

In a more preferred embodiment of the invention the agonist is a full, partial or inverse agonist. Full agonists activate the receptor to a maximal extent. Compounds having a lower effect than a full agonist are called partial agonist, since they stimulate signal transduction but to a lesser extent than a full agonist. An inverse agonist is a ligand which produces an effect opposite to that of the agonist by occupying the same receptor. Especially in systems in which the G protein coupled receptor is over-expressed inverse agonists can be identified. They reduce the basal level of the measurable or detectable signal upon binding to the receptor.

The identified ligands can be used to elucidate the characteristics and function of the APJ receptor. Furthermore, the ligands are potential drugs useful in treatment and prevention of APJ receptor related disorders or disease, preferably heart diseases, most preferably congestive dilated cardiomyopathy, hypertrophic cardiomyopathy and coronary artery disease.

The term “APJ receptor” intends a receptor for apelin. An APJ receptor according to the present invention is any naturally occurring APJ receptor. This includes APJ receptors and variants thereof of different species, preferably vertebrates, more preferably mammals, as well as splice variants or APJ receptors occurring due to receptor polymorphism as known for other receptors such as β₂-adrenergic receptor. Preferred mammalian APJ receptors or variants thereof include human (FIG. 2), mouse (Mus musculus, WO 00/68244) and rat (Rattus norvegicus, WO 00/68250) APJ receptor. The human APJ receptor or variants thereof is the most preferred APJ receptor. In any case the receptor must be able of transducing a signal, i.e. modifying a Gαq or a Gαi protein or a hybrid protein which is assembled from portions of different G proteins like Gαq or Gαi proteins upon ligand binding. “Modifying” according to the present invention means activating or inactivating a Gαq or a Gαi protein or a hybrid protein which is assembled from portions of different G proteins like Gαq or Gαi proteins. In general the receptor activates a Gαq or a Gαi protein or a hybrid protein which is assembled from portions of different G proteins like Gαq or Gαi proteins upon agonist binding. In case of an activated receptor, e.g. a constitutive active variant of the APJ receptor, the receptor inactivates a Gαq or a Gαi protein or a hybrid protein which is assembled from portions of different G proteins like Gαq or Gαi proteins upon antagonist binding.

A variant of the APJ receptor according to the present invention is an APJ receptor which binds apelin but does not naturally occur. The binding of apelin to the variant of the APJ receptor can be examined by using the apelin analogue [¹²⁵I]-Pyr¹)Apelin-13 as radioligand and performing radioligand binding experiments as described by Katugampola and colleagues (Katugampola et al, 2001, Br J Pharmacol 132: 1255-1260). The term “variant” is used to refer to an oligonucleotide sequence which differs from the naturally occurring APJ receptor sequence in one or more nucleotides. Such a variant oligonucleotide is expressed as a protein variant which, as used herein, indicates a polypeptide sequence that differs from the wild-type polypeptide (APJ receptor) in the substitution, insertion or deletion of one or more amino acids. Preferred are conservative amino acid substitutions, whereby a residue is substituted by another with like characteristics. Typical substitutions are among the aliphatic amino acids, among the amino acids having aliphatic hydroxyl side chain, among the amino acids having acidic residues, among the amide derivatives, among the amino acids with basic residues, or the amino acids having aromatic residues. The variant polypeptide differs in primary structure (amino acid sequence), but may or may not differ significantly in secondary or tertiary structure or in function relative to the wild-type. The variant can also be a portion of the APJ receptor sufficient for binding of apelin or a receptor chimera including an APJ receptor portion fused to a second G protein coupled receptor portion, wherein the chimera is still able of binding apelin and the second G protein coupled receptor portion is a portion of any other G protein coupled receptor. In any case the receptor must be able of transducing a signal, i.e. modifying a Gαq or a Gαi protein or a hybrid protein which is assembled from portions of different G proteins like Gαq or Gαi proteins (e.g. G_{αΔ6qi4myr} protein), upon ligand binding.

Three G-proteins have been tested in the APJ assay and could be used for the detection of APJ ligands: G_{αΔ6qi4myr}) G_{α16}, and G_{αi4qi4}. The G_{αΔ6qi4myr} protein is (FIG. 1) disclosed in WO 02/04665 (G_{α-6qi4myr}, SEQ ID NO: 2). This G protein is a hybrid G protein assembled from portions of different G proteins containing additional modifications and combines sequences of various α subunits within one protein. This fusion protein of different G proteins was designed by fusing the G_{α} receptor recognition region of one G protein to the G_{α} effector activation region of another G protein. The aim was to provide a G protein hybrid which receives signals from G protein coupled receptors but switches on the signal transduction pathway to which the receptor is naturally not coupled to. This “recoupling” of receptors has the advantage that the assay endpoint (increase in intracellular Ca²⁺ concentration in comparison with adenylyl cyclase inhibition) is more readily accessible through measurement methods and can be used in high throughput screening. To build the G_{αΔ6qi4myr} protein the 6 highly conserved N-terminal amino acids of the G_{αq} protein were deleted. Additionally, to the lack of the six highly conserved amino acids of the amino terminus the hybrid has a G_{αi} protein sequence at the C terminus. Finally, additional myristoylation/palmitoylation recognition sequences were inserted into the amino-terminal region of the G_{α} subunit to produce G_{-6qi4myr} protein (i.e. G_{αΔ6qi4myr} protein). The protein sequence of −6qi4myr at the amino terminus is MGCC, in contrast to MACC in the original sequence of the −6q variants. The G_{αi4qi4} protein is also covered in WO02/04665. This G protein is a hybrid G protein which is assembled from portions of different G proteins containing additional modifications and combines sequences of various Gα protein subunits within one protein. For building up the G_{αi4qi4} protein the six highly conserved amino acids of the G_{αq} protein were deleted. Additionally, to the lack of the six highly conserved amino acids of the amino terminus the first four amino acids (MACC) have been substituted by the first four amino acids of G_{αi} protein (MGCT). Furthermore the G_{αi4qi4} protein has a G_{αi} protein sequence at the C terminus (CGLF). The G_{α16} protein is disclosed in Offermann et al., (1995) J. Biol. Chem 270, 15175 - 15180 (Ga15 and Ga16 couple a wide variety of receptors to phospholipase C). The measurable or detectable signal is produced by an effector, which is a molecule down-stream from the hybrid G proteins (G_{αΔ6qi4myr}, G_{αi4qi4} ) and the G_{α16} protein in the signal transduction pathway. A measurable or detectable signal according to the present invention is any change of concentration of a substance or charging which is mediated upon ligand binding to the APJ receptor. The signal can be for example a concentration change of an intracellular compound, e.g. a second messenger such as diacylglycerol or inositol 1,4,5-trisphosphate or ion such as Ca²⁺. The signal can be measured by any known method using selective electrodes, antibodies, radiolabels, fluorescence markers, enzymes and so on.

In general the hybrid G proteins (G_{αΔ6qi4myr}, G_{αi4qi4} ) and the G_{α16} protein couple to various phospholipase Cβ isoforms, which leads to hydrolysis of membrane-bound phosphatidylinositol 4,5-bisphosphate to give diacylglycerol (DAG) and inositol 1,4,5-trisphosphate (IP₃). IP₃ releases Ca²⁺ from intracellular depots. Therefore, measurable or detectable signals include changes of DAG, IP₃ and Ca⁺.

Methods for the detection and quantification of DAG are known to the artisan. DAG in crude lipid extracts can be measured by the method of Preiss et al (Preiss et al, 1986, J Biol Chem 261: 8597-8600). Cells are stimulated with a putative APJ agonist and solubilized. Produced DAG is reacted with radiolabelled ATP in the presence of DAG kinase, isolated by chromatography and quantified by counting in a β- or γ-counter. Suitable markers for the ATP include ³H, ³²P, ³³P, ³⁵S (when using e. g. adenosine thiotriphosphate) and ¹⁴C.

Methods for the detection and quantification of IP₃ are known to the one of skill in the art. IP₃ can be measured using commercial available radioimmoassays (e.g. Amersham, Ill., USA) according to the manufacturers' instructions. Another method of measuring IP₃ includes the incubation of cells with labelled inositol, stimulation of the cell with a putative APJ receptor agonist, lysis of the cells, anion exchange column separation, and quantification of produced labelled IP₃ (Berridge, 1983, Biochem J 212: 849-858). Suitable markers for inositol include ³H and ¹⁴C.

In a more preferred embodiment the signal to be measured or detected is a change of Ca²⁺ concentration, most preferably of intracellular Ca²⁺ concentration, which is to be measured or detected easily and which can be utilized in a screening assay with high sample throughput.

Suitable methods for the detection and quantification of Ca²⁺ are known to the artisan (Takahashi et al., 1999, Physiol Rev 79: 1089-1125) and include but are not limited to methods using calcium-sensitive probes. Ca²⁺ is in general measured using fluorescent dyes. There exist several well known protein based and non-protein based Ca²⁺ indicators including aequorin, modified green fluorescent protein-calmodulin chimer, Quin, Indo, Fura, BTC, BAPTA etc. All these methods and fluorescent dyes are well known to the one of ordinary skill in the art.

In general cells are grown and loaded with a fluorescence marker and then stimulated with a putative APJ agonist. Thereafter Ca²⁺ is measured with a fluorescence photometer at particular excitation wavelength and emission wavelengths which depend on the used marker.

In the most preferred embodiment the Ca²⁺ is measured by a FLIPR (Fluorometric Imaging Plate Reader) apparatus (Molecular Devices, Calif., USA) which typically measures intracellular Ca²⁺ levels in 96-well and 384-well formats. The assay is performed according to the manufacturer's instructions. This method of measuring Ca²⁺ is highly suitable for high through put assays.

In one embodiment of the invention the test system is located in a genetically engineered cell, into which at least one of the components (a) or (b) (an APJ receptor or a functional variant thereof, a G_{αΔ6qi4myr} protein) is introduced. The introduced component is referred to as transgene.

These cells include but are not limited to HEK 293, 745-A, A-431, atrial myocytes, BxPC3, C5N, Caco-2, Capan-1, CC531, CFPAC, CHO, CHO K1, COS-1, COS-7, CV-1, EAHY, EAHY 926, F98, GH3, GP&envAM12, H-295 R, H-4-II-E, HACAT, HACAT A131, HEK, HEL, HeLa, Hep G2, High Five, Hs 766T, HT29, HUV-EC R24, HUV-EC-C, IEC 17, IEC 18, Jurkat, K 562, KARPAS-299, L 929, LIN 175, MAt-LYLU, MCF-7, MNEL, MRC-5, MT4, N64, NCTC 2544, NDCK II, Neuro 2A, NIH 3T3, NT2/D1, P19, primary neuronal cells, primary dendritic cells, primary human myoblasts, primary keratinocytes, SF9, SK-UT-1, ST, SW 480, SWU-2 OS, U-373, U-937, and Y-1. Other suitable cells are those known to the one of skill in the art.

Preferred cell lines are HEK 293 cells (primary human embryonal kidney), 3T3 cells (murine embryonal fibroblasts), CHO cells (Chinese hamster ovary), COS-7 cells (African green monkey cell line), HeLa cells (human epithelioid cervical carcinoma), JURKAT cells (human T-cell leukaemia), BHK 21 cell (hamster normal kidney, fibroblast), and MCF-7 cells (human breast cancer).

In another embodiment of the invention the test system is located in a transient or stable transfected cell line or membrane thereof. The procedure for introducing a transgene into a recipient cell is called transfection. Transfection with DNA yields stable as well as unstable (transient) cell lines. Transient cell lines reflect the survival of the transfected DNA in extrachromosomal form; stable cell lines result from the integration into the genome. In case the measuring and detecting of the signal is not limited to whole cells, isolated membranes of such cell lines can be used. Isolated membranes are e.g. provided by harvesting cells, optionally homogenising them and collecting them by centrifugation.

The transgenes can be introduced into the cells by a variety of means known to those knowledgeable in the art, and adapted to each cell type. Recombinant DNA cloning techniques well known in the art for introducing and expressing a nucleic acid molecule can be used to introduce and express the transgenes. Cells can be transfected using any appropriate means, including viral vectors, chemical transfectants, electroporation, calcium phosphate co-precipitation and direct diffusion of DNA.

As used herein, vectors are agents that transport the transgene into the cell and may include appropriate transcriptional and translational control signals such as a promoter. Vectors can be plasmid, viral or others known in the art. The promoter can be inducible or constitutive, general or cell specific, nuclear or cytoplasmic specific promoter. Selection of promoters, vectors and other elements is a matter of routine design within the level of ordinary skill in the art. Many such elements are described in the literature and are available through commercial suppliers. Usually, the method of transfer includes the transfer of a selectable marker to the cells.

In general a cell line is transfected by any of the means mentioned above wherein the transgene is operatively linked to a selectable marker. Following transfection cells are grown e. g. for some days in enriched media and then switched to selective media. Transfected cells exhibit resistance to the selection and are able to grow, whereas non-transfected cells die in general. Examples for selective markers include puromycin, zeocin, neomycin (neo) and hygromycin B, which confer resistance to puromycin, zeocin, aminoglycoside G-418 and hygromycin, respectively.

Another subject of the invention is a method of screening an APJ receptor ligand, wherein the method comprises the steps of:
(a) providing the test system according to present invention,
(b) providing a test compound,
(c) optionally providing a known APJ receptor ligand, and
(d) measuring or detecting the influence of the test compound on the APJ receptor mediated signal transduction pathway.

The aim of the method of screening is the identification of a test compound as an APJ receptor ligand and its characterisation. The test compound can be any test compound either naturally occurring or chemically synthesized. Naturally occurring test compounds include in particular peptides, preferably peptides showing similarity to the endogenous ligand apelin. This includes apelin fragments as well as peptides having a particular sequence homology (the higher the homology the higher the change of identifying an APJ receptor agonist). But the test compound may also be a non-proteinous ligand. It is a fact that the seven transmembrane domains of the G protein coupled receptors form a binding pocket. It is a matter of common knowledge that non-peptide ligands such as catecholamines (epinephrine, norepinephrine) bind more interiorly in the binding pocket than peptides do (their binding site is more outwards). Consequently a suitable target may also be a smaller (as compared to the peptide ligands) non-peptide compound. It is expected that such smaller compounds exhibit upon binding to the receptor preferably antagonist properties as it is known for the angiotensin receptor (losartan, candesartan).

Depending on the assay design agonists, preferably, full, partial or inverse agonists, and antagonists can be identified.

For the identification of full, partial and inverse agonists the method of screening can be performed carrying out steps (a), (b) and (d). For this the test system is incubated with the test compound in a suitable environment (buffer, temperature, substrate etc.) and for a time sufficient to detect or measure a signal. If the test compound is any kind of agonist the concentration should be high enough to induce a signal. The concentration should not exceed the toxicity level which is in general between 0.1 and 1 mol/l.

If an agonist was identified it can be further characterized by using the test system. Full and partial agonist can be characterized by dose response curves. Hereunto, the test system is incubated with increasing concentrations of the test compound. The signal is plotted against the concentration of the agonist. The resulting curve can be analyzed using the in the following equation:
S([C])=Sₘᵢₙ+(Sₘₐₓ−Sₘᵢₙ)·[C]/([C]+EC₅₀)
[C]=concentration of the test compound C
S([C])=signal at a particular concentration of test compound C
Sₘᵢₙ=basal or minimal signal
Sₘₐₓ=maximal signal
EC₅₀=concentration of test compound C mediating a half maximal signal

The test compound is then characterized by the EC₅₀ and Sₘₐₓ. If Sₘₐₓ is as high as Sₘₐₓ induced with a full agonist the test compound is also a full agonist. If it is lower, the test compound is a partial agonist. In general drugs with low EC₅₀ values are preferred, since less substance is needed for the same effect as compared to a drug with a higher EC₅₀ value.

For the identification of antagonists the method of screening can be performed carrying out steps (a) to (d). Since antagonists in general do not mediate the production of a measurable or detectable signal they cannot be identified directly. Therefore, the binding of an antagonist is identified by blocking the effect of an agonist ((c) known APJ receptor ligand). The test system is incubated with the test compound and an agonist in a suitable environment (buffer, temperature, substrate etc.) and for a time sufficient to detect or measure a signal. If the test compound is any kind of antagonist the concentration should be high enough to inhibit the signal mediated by the agonist. The concentration of the test compound and the agonist should not exceed the toxicity level which is in general between 0.1 and 1 mol/l.

If an antagonist was identified it can be further characterized by using the test system. Hereunto, the test system is incubated with increasing concentrations of the test compound in the presence of a defined concentration of an agonist. The signal is plotted against the concentration of the antagonist. The resulting curve can be analyzed using the in the following equation:
S([B])=Sₘᵢₙ+(Sₘₐₓ−Sₘᵢₙ){1−[B]/([B]+IC₅₀)}
[B]=concentration of the test compound B (blocker=antagonist)
S([B])=signal at a particular concentration of test compound B
Sₘᵢₙ=basal or minimal signal
Sₘₐₓ=maximal signal
IC₅₀=concentration of test compound B mediating a half maximal inhibition
   The blocker constant K_{B} can be calculated according to:
   K_{B}=IC₅₀{1+[A]/EC_{50}}
[A]=concentration of the agonist
EC₅₀=concentration of the agonist mediating a half maximal signal

The test compound is then characterized by the K_{B}. In general drugs with low K_{B}-values are preferred, since less substance is needed to block or inhibit the same signal.

In one preferred embodiment of the invention the test compound is provided in the form of a chemical compound library. Chemical compound libraries include are plurality of chemical compounds and have been assembled from any of multiple sources, including chemically synthesized molecules and natural products, or have been generated by combinatorial chemistry techniques. They are especially suitable for high throughput screening. They may be comprised of chemical compounds of a particular structure or compounds of a particular creature such as a plant. In the context with the present invention the chemical compound library is preferably a library comprising proteins and polypeptides or ligands for G protein coupled receptors.

In one preferred another embodiment of the invention the method of screening a ligand is carried out using whole cells. The cells can be any known in the art. Examples of useful cells are mentioned above. Usually cells are grown in multiwell plates until use. Cells are optionally labelled (if necessary) and subsequently stimulated with an APL receptor ligand. Using whole cells is advantageous in contrary to membranes, since intracellular substrates, enzymes etc. need not to be added to the test system. Furthermore, whole cells in multiwell plates are particularly suitable for high trough put screening test and automated test systems.

Advantageously the method of the present invention is carried out in a robotics system e.g. including robotic plating and a robotic liquid transfer system, e.g. using microfluidics, i.e. channelled structured.

In another embodiment of the present invention, the method is carried out in form of a high-through put screening system. In such a system advantageously the screening method is automated and miniaturized; in particular it uses miniaturized wells and microfluidics controlled by a roboter.

Another subject of the present invention is a method for producing a medicament, wherein the method comprises the steps of:
(a) identifying an APJ receptor ligand carrying out the method of screening according to the present invention,
(b) providing adequate amounts of the ligand, and
(c) formulating the measured or detected test compound with one or more pharmaceutically acceptable carriers or auxiliary substances.

The medicament is preferably used for the prophylaxis, therapy or diagnosis of an APJ-receptor related disease or disorder. An APJ receptor-related disease or disorder is a condition characterized in that the expression level of the receptor, of its endogenous ligand, its the signal transduction, its signal transduction pathway or the effects of an down-stream effector molecule is changed. Alternatively, the APJ receptor is involved in the development or progression of a disease or disorder. Examples of such diseases include, but are not limited to, cardiovascular diseases, heart diseases, malfunction of blood pressure regulation, diseases of the nervous system and the immune system, such as HIV.

More preferably, the disease is a heart disease, most preferably congestive dilated cardiomyopathy, hypertrophic cardiomyopathy and coronary artery disease.

For the medication the isolated test compound or its pharmaceutically acceptable salt has to be in a pharmaceutical dosage form in general consisting of a mixture of ingredients such as pharmaceutically acceptable carriers or auxiliary substances combined to provide desirable characteristics.

The formulation comprises at least one suitable pharmaceutically acceptable carrier or auxiliary substance. Examples of such substances are demineralised water, isotonic saline, Ringer's solution, buffers, organic or inorganic acids and bases as well as their salts, sodium chloride, sodium hydrogencarbonate, sodium citrate or dicalcium phosphate, glycols, such a propylene glycol, esters such as ethyl oleate and ethyl laurate, sugars such as glucose, sucrose and lactose, starches such as corn starch and potato starch, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils such as groundnut oil, cottonseed oil, corn oil, soybean oil, caster oil, synthetic fatty acid esters such as ethyl oleate, isopropyl myristate, polymeric adjuvans such as gelatin, dextran, cellulose and its derivatives, albumins, organic solvents, complexing agents such as citrates and urea, stabilizers, such as protease or nuclease inhibitors, preferably aprotinin, ε-aminocaproic acid or pepstatin A, preservatives such as benzyl alcohol, oxidation inhibitors such as sodium sulphite, waxes and stabilizers such as EDTA. Colouring agents, releasing agents, coating agents, sweetening, flavouring and perfuming agents, preservatives and antioxidants can also be present in the composition. The physiological buffer solution preferably has a pH of approx. 6.0-8.0, especially a pH of approx. 6.8-7.8, in particular a pH of approx. 7.4, and/or an osmolarity of approx. 200-400 milliosmol/liter, preferably of approx. 290-310 milliosmol/liter. The pH of the medicament is in general adjusted using a suitable organic or inorganic buffer, such as, for example, preferably using a phosphate buffer, tris buffer (tris(hydroxymethyl)aminomethane), HEPES buffer ([4-(2-hydroxyethyl)piperazino]ethanesulphonic acid) or MOPS buffer (3-morpholino-1-propanesulphonic acid). The choice of the respective buffer in general depends on the desired buffer molarity. Phosphate buffer is suitable, for example, for injection and infusion solutions. Methods for formulating a medicaments as well as suitable pharmaceutically acceptable carrier or auxiliary substance are well known to the one of skill in the art. Pharmaceutically acceptable carriers and auxiliary substances are a. o. chosen according to the prevailing dosage form and identified compound.

The pharmaceutical composition can be manufactured for oral, nasal, rectal, parenteral, vaginal, topic or vaginal administration. Parental administration includes subcutaneous, intracutaneous, intramuscular, intravenous or intraperitoneal administration.

The medicament can be formulated as various dosage forms including solid dosage forms for oral administration such as capsules, tablets, pills, powders and granules, liquid dosage forms for oral administration such as pharmaceutically acceptable emulsions, microemulsions, solutions, suspensions, syrups and elixirs, injectable preparations, for example, sterile injectable aqueous or oleaginous suspensions, compositions for rectal or vaginal administration, preferably suppositories, and dosage forms for topical or transdermal administration such as ointments, pastes, creams, lotions, gels, powders, solutions, sprays, inhalants or patches.

The specific therapeutically effective dose level for any particular patient will depend upon a variety of factors including the activity of the identified compound, the dosage form, the age, body weight and sex of the patient, the duration of the treatment and like factors well known in the medical arts.

The total daily dose of the compounds of this invention administered to a human or other mammal in single or in divided doses can be in amounts, for example, from about 0.01 to about 50 mg/kg body weight or more preferably from about 0.1 to about 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of the compounds of the present invention per day in single or multiple doses.

Still another subject of the present invention is a method for diagnosis or prognosis for a heart disease comprising the following steps:
(a) determining the expression level of APJ receptor gene and/or apelin gene in a sample to be tested,
(b) comparing the expression level of APJ receptor gene and/or apelin gene in the sample to be tested determined in step (a) with the expression level of APJ receptor gene and/or apelin gene in a control probe, and
(c) identifying a sample with increased APJ receptor and/or apelin gene expression.

The APJ receptor and apelin can be used as indices of a heart disease, preferably congestive dilated cardiomyopathy, hypertrophic cardiomyopathy and coronary artery disease. Surprisingly the inventors found that the expression of APJ receptor as well as apelin was increased in diseased heart (table 1). Therefore, an increased expression of APJ receptor and apelin is obviously an indicator for heart diseases such as congestive dilated cardiomyopathy, hypertrophic cardiomyopathy and coronary artery disease.

In the method for diagnosis or prognosis according to the present invention, expression of the APJ receptor gene or the apelin gene can be assayed by a nucleic acid amplification method, hybridization method, enzyme immunoassay, fluorescence immunoassay, luminescent immunoassay or protein bioassay which include membrane, solution, or chip-based technologies for the detection and/or quantification of nucleic acid or protein and so on.

In a preferred embodiment the method for diagnosis and prognosis is carried out by determining the expression levels of the gene(s) on the basis of levels of mRNAs transcribed from the gene(s) or levels of polypeptides translated from the genes.

A wide variety of labels and conjugation techniques are known by those skilled in the art and can be used in various nucleic acid and amino acid assays. Means for producing labelled hybridization or PCR probes for detecting sequences related to polynucleotides encoding APJ receptor or apelin include oligolabelling, nick translation, end-labelling, or PCR amplification using a labelled nucleotide. Alternatively, sequences encoding APJ receptor or apelin can be cloned into a vector for the production of an mRNA probe. Such vectors are known in the art, are commercially available, and can be used to synthesize RNA probes in vitro by addition of labelled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

A variety of techniques known in the art can be used to quantify the level at which a given polypeptide or protein is expressed. These include, but are not limited to immunological techniques such as an ELISA, RIA, or western blot, or quantitative analytical techniques such as spectroscopy or flame chromatography.

In a more preferred embodiment the method for diagnosis and prognosis is carried out by determining the levels of mRNAs by a hybridization method or a nucleic acid amplification method. Such methods are known to the artisan and include the dot blot hybridization method, the Northern hybridization method or the RT-PCR method.

In another more preferred embodiment the method for diagnosis and prognosis is carried out by determining the levels of polypeptides by using binding proteins, e.g. antibodies or anticalins, capable of binding specifically to the polypeptides or fragments thereof.

A variety of protocols for detecting and measuring polypeptides using either polyclonal or monoclonal antibodies specific for the polypeptide, are known in the art. Examples include enzyme-linked immunosorbent assay (ELISA), radioimmunoassay (RIA), and fluorescence activated cell sorting (FACS).

For this method the antibodies capable of binding specifically to polypeptides encoded by the APJ receptor gene and/or apelin gene for carrying out the prevailing test (see above) are used. The procedure for preparing an antibody or antibody fragment is effected in accordance with methods which are well known to the skilled person, e.g. by immunizing a mammal, for example a rabbit, with the APJ receptor or apelin, where appropriate in the presence of, for example, Freund's adjuvant and/or aluminium hydroxide gels (see, for example, Diamond, B. A. et al. (1981) The New England Journal of Medicine: 1344-1349). The polyclonal antibodies which are formed in the animal as a result of an immunological reaction can subsequently be isolated from the blood using well known methods and, for example, purified by means of column chromatography. Monoclonal antibodies can, for example, be prepared in accordance with the known method of Winter & Milstein (Winter, G. & Milstein, C. (1991) Nature, 349, 293-299).

Another subject of the present invention is a kit usable for detection or prognosis of a heart disease, comprising primers and/or a probe which is usable for determining expression levels of the APJ receptor gene and/or apelin gene.

The kit usable for detection and prognosis of a heart disease includes a kit for examining alterations in the expression levels of the APJ receptor and/or apelin gene in a sample to be tested. The kit of the present invention is a kit capable of quantifying the level of mRNA transcribed from the above-mentioned genes. In order to examine expression the kit comprises primers and/or a probe which is usable for determining the level of mRNA from the gene.

The kit of the present invention is based on the surprising findings that APJ receptor and apelin gene expression is increased in the hearts of patients with heart disease, in particular congestive dilated cardiomyopathy, hypertrophic cardiomyopathy and coronary artery disease.

The primers are designed to specifically bind to nucleic acids corresponding to the above-mentioned genes. They include any primers which are capable of specifically amplifying nucleic acid sequences derived from mRNA transcribed from the above-mentioned genes under reaction conditions used for any conventional nucleic acid amplification method, e.g. RT-PCR. For instance, primers can be designed and synthesized on the basis of the nucleotide sequences of these genes. In an preferred embodiment the kit comprises a pair of primers capable of specifically amplifying a nucleic acid sequence derived from mRNA transcribed from the above-mentioned genes. Furthermore the kit may comprise suitable reagents for carrying out the amplification the mRNA and quantification of the gene expression.

Another subject of the present invention is a kit usable for detection or prognosis of a heart disease, comprising antibodies capable of binding specifically to polypeptides encoded by the APJ receptor gene and/or apelin gene.

A variety of techniques known in the art can be used to quantify the level at which a given protein is expressed. For further details see above.

TABLE 1
Comparison of Gene Expression in Diseased and Normal Nonfailing Heart

Gene expression
(% of total samples)

| | APJ | |
|---|---|---|
| | receptor | Apelin |
| Normal nonfailing heart (n = 52) | 26* [70] | 15* [30] |
| Diseased heart (n = 462) | 92* [465] | 64* [61] |
| Congestive dilated cardiomyopathy (n = 124) | 95* [465] | 62* [55] |
| Coronary artery disease (n = 301) | 92* [410] | 63* [63] |
| Hypertrophic cardiomyopathy (n = 37) | 83* [506] | 72* [66] |

*Indicates the % of samples in given sample set wherein the gene was determined to be expressed. The median expression value (in arbitrary units) for sample set is given in [].

### DESCRIPTION OF THE FIGURES

FIG. 1 depicts the polynucleotide sequence of the mouse G_{αΔ6qi4myr} protein coding region inserted into pCDNA 1 (corresponding to Seq ID No. 1)

The G_{αΔ6qi4myr} protein gene was inserted into pCDNA 1 5′ and 3′ via a BamHI and a NsiI site, respectively.

FIG. 2 shows the human apelin receptor expression plasmid and the amino acid sequence of the receptor protein (corresponding to Seq ID No. 2)

The human apelin receptor gene was cut from AVEM1139400 and inserted into pEAK8 which was cut with EcoRI.

FIG. 3 shows the effects of various apelin peptides on the APJ receptor in transiently cotransfected CHO cells

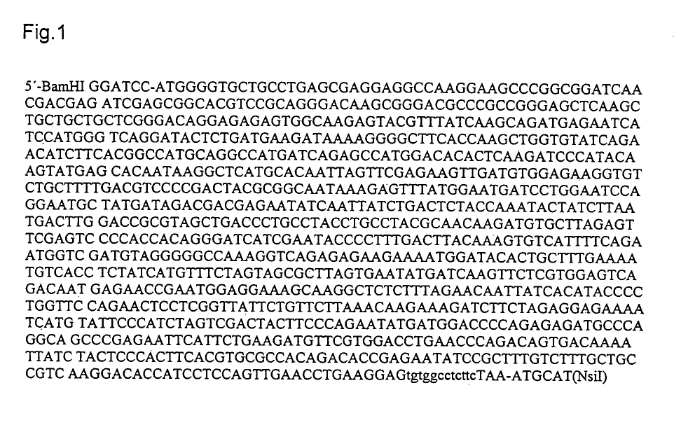

The APJ receptor was transiently cotransfected in CHO cells (Chinese hamster ovary cells) with vector DNA as a control (white column) or the G protein α subunits α16 (black column) and αΔ6qi4myr (striped column) according to methods known to the one skilled in the art. Cells were challenged with 1 μmol/l of peptide each:
1 apelin like peptide/jerini peptide
2 apelin 12
3 apelin 13
4 apelin 36
5 pyr-apelin
   and the intracellular Ca²⁺ concentration determined in a functional FLIPR calcium assay. Peptides 2-5 induced intracellular calcium release via the APJ receptor. It was shown that functional calcium responses required the presence of a chimeric/promiscuous G protein α subunit. Data presented are mean values (fluorescence intensity units, FIU) from 2 individual experiments performed in duplicate.

FIG. 4 shows dose-dependently the effects of apelin on the APJ receptor in stably /transfected HEK 293 cells overexpressing G_{αi4qi4} and APJ receptor

HEK 293 cells were stably transfected with G_{αΔ6qi4myr} alone (A) or cotransfected with G_{αΔ6qi4myr} and APJ (B) as described above and incubated with

1 none
   2 50 nmol/l
3 200 nmol/l
4 1 μmol/l
   pyr-apelin-13 (top) or apelin-36 (bottom). Both peptides induced intracellular calcium release via the APJ receptor in a dose-dependent manner. It was shown that functional calcium responses required the presence of the APJ receptor. Data presented are mean values (fluorescence intensity units, FIU) from 2 individual experiments performed in duplicate.

FIG. 5 depicts the amino acid sequence of G_{αΔ6qi4myr} protein (corresponding to Seq ID No. 3)

FIG. 6 depicts the polynucleotide sequence of the G_{αi4qi4} gene coding region (corresponding to Seq ID No. 4)

FIG. 7 depicts the amino acid sequence of G_{αi4qi4} protein (corresponding to Seq ID. No. 5)

FIG. 8 depicts the amino acid sequence of human apelin-12 (corresponding to Seq ID No. 6)

FIG. 9 depicts the amino acid sequence of human apelin-13 (corresponding to Seq ID No. 7).

FIG. 10 depicts the amino acid sequence of apelin-36 (corresponding to Seq ID No. 8).

Description of Seq IDs:

Seq ID No. 1: Polynucleotide sequence of mouse G_{αΔ6qi4myr} protein coding region inserted into pCDNA 1

Seq ID No. 2: Polynucleotide sequence of human apelin receptor expression plasmid (human apelin receptor gene was cut from AVEM1139400 and inserted into pEAK8 which was cut with EcoRI) and the amino acid sequence of the receptor protein.

Seq ID No.3: Amino acid sequence of mouse G_{αΔ6qi4myr} protein.

Seq ID No.4: Polynucleotide sequence of mouse G_{αi4qi4} gene coding region.

Seq ID No. 5: Amino acid sequence of mouse G_{αi4qi4} protein.

Seq ID No. 6: Amino acid sequence of human apelin-12.

Seq ID No. 7: Amino acid sequence of human apelin-13.

Seq ID No. 8: Amino acid sequence of human apelin-36.

### EXAMPLES

1. Functional APJ receptor/G_{αi4gi4} Protein Cell-Line

1.1. Cell Culture

HEK 293 cells (human embryo kidney cells) were grown in DMEM (Dulbecco's modified Eagle's medium) with 10% FCS (fetal calf serum) at 37° C. (5% CO₂). After the second transfection the antibiotics zeocin and puromycin were added to the cells. Cells were passaged at two- or three-day intervals.

1.2. Transfection of a HEK293 cell line with the G_{αi4qi4} Protein

For transfection cells were seeded in 100-mm plates. About 24 hours later, the cells were transfected with the pHOOK3 vector (Invitrogen, MD, USA) carrying the G_{αi4qi4} (FIG. 6). Stabile cell lines were stored frozen until use.

1.3. Transfection of the Cell Line with the APJ Receptor

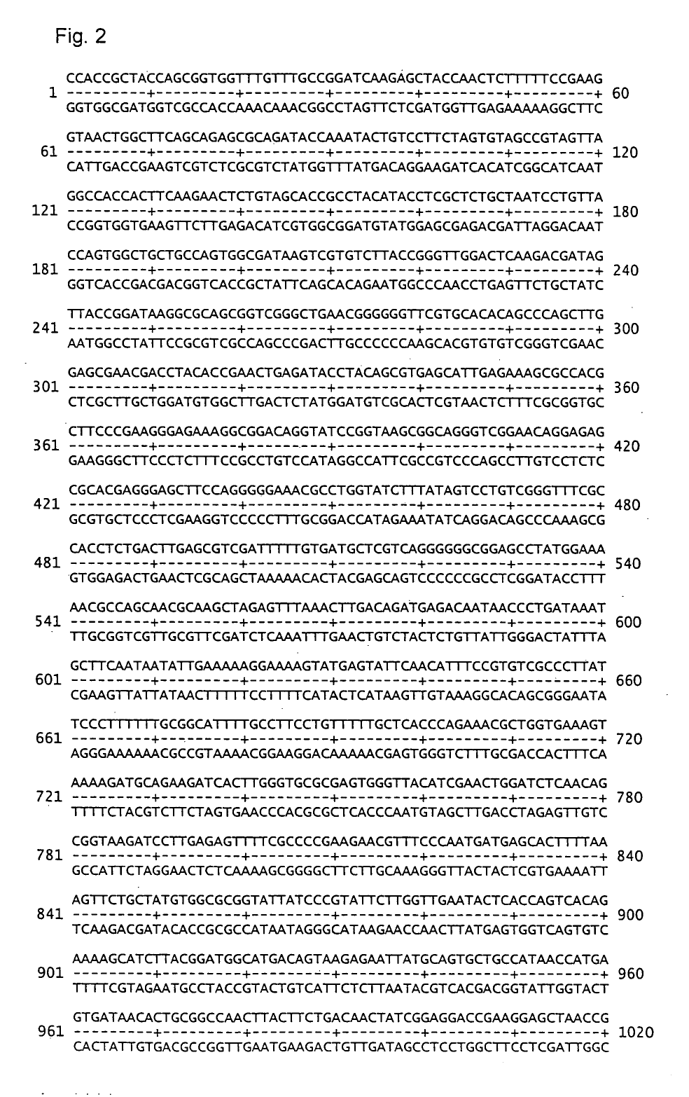

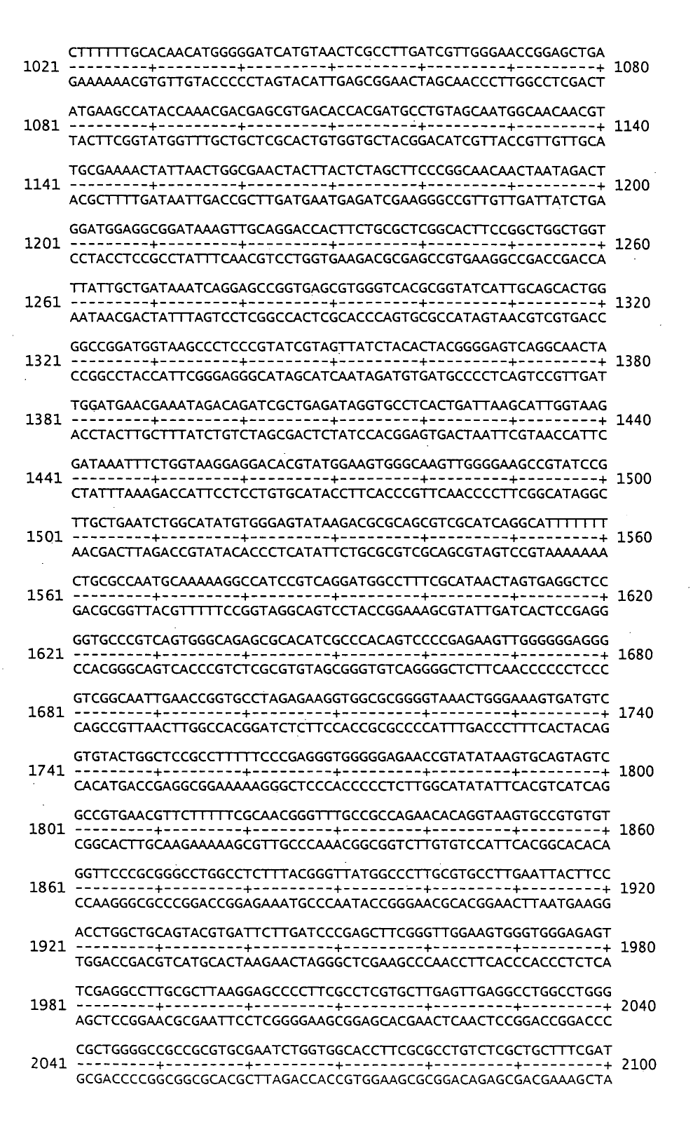

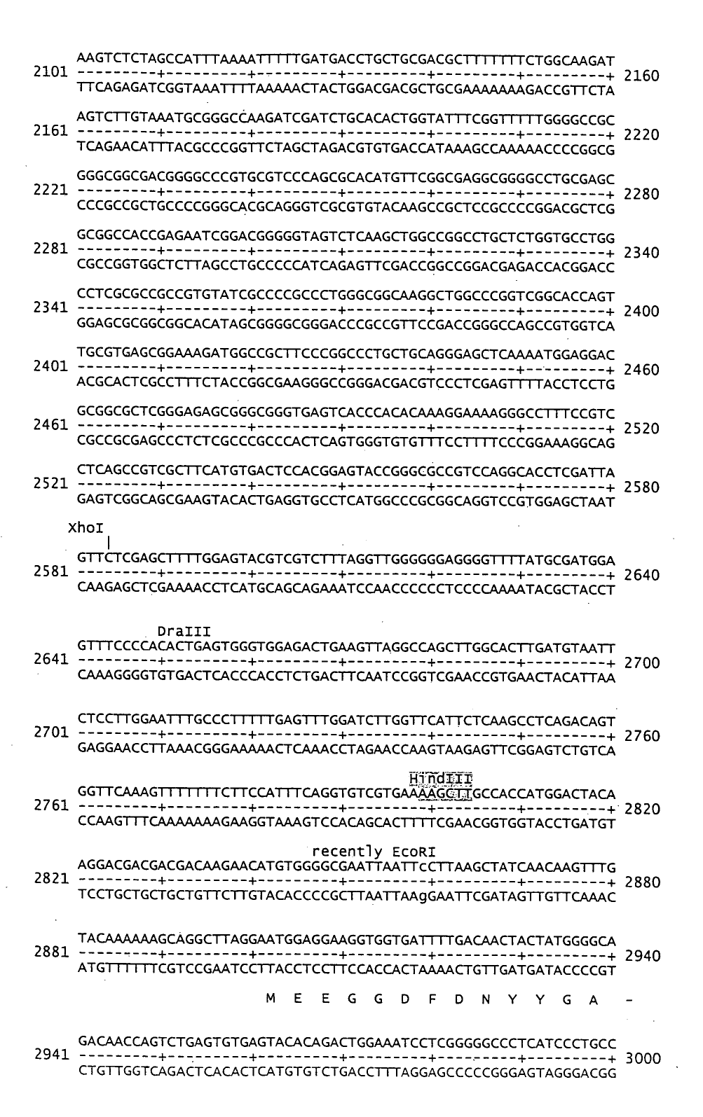

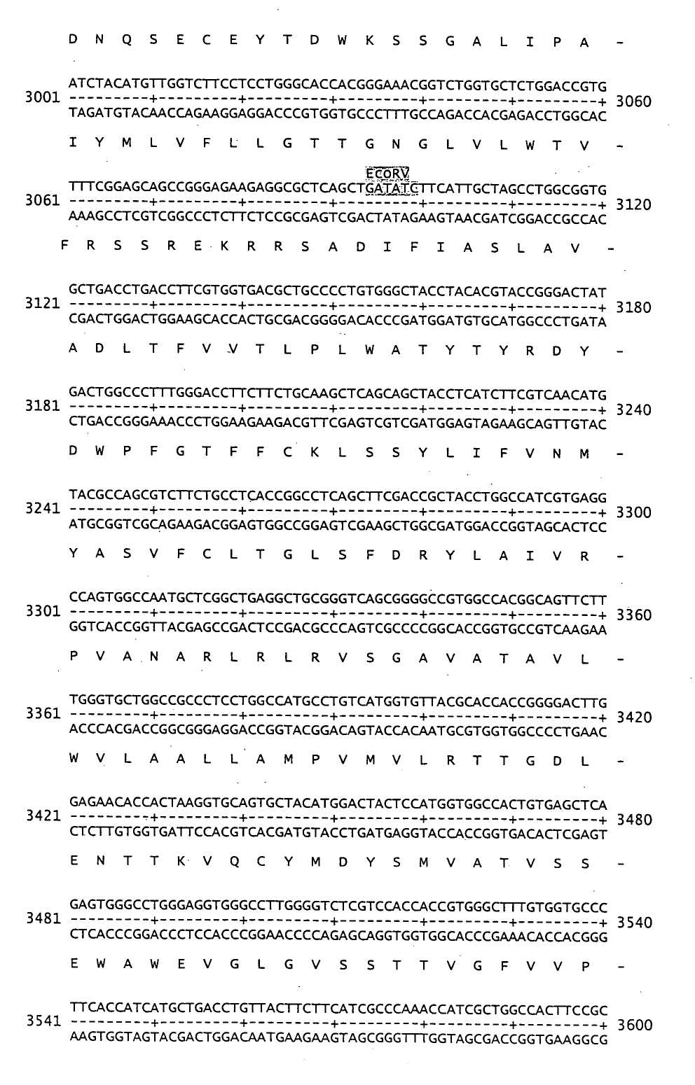

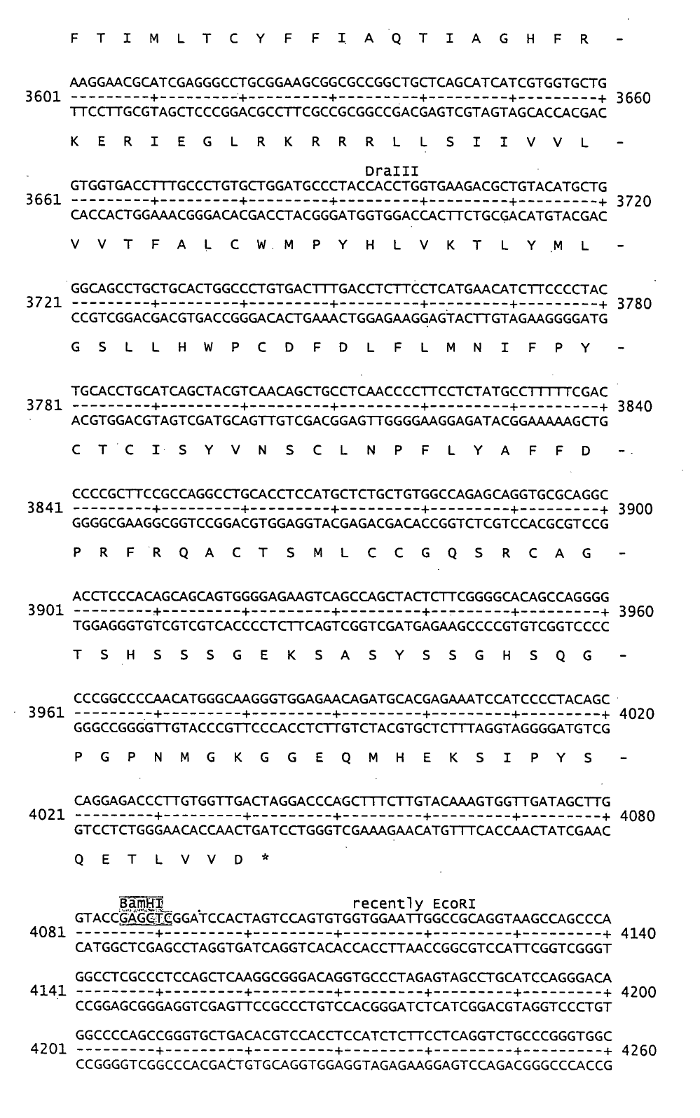

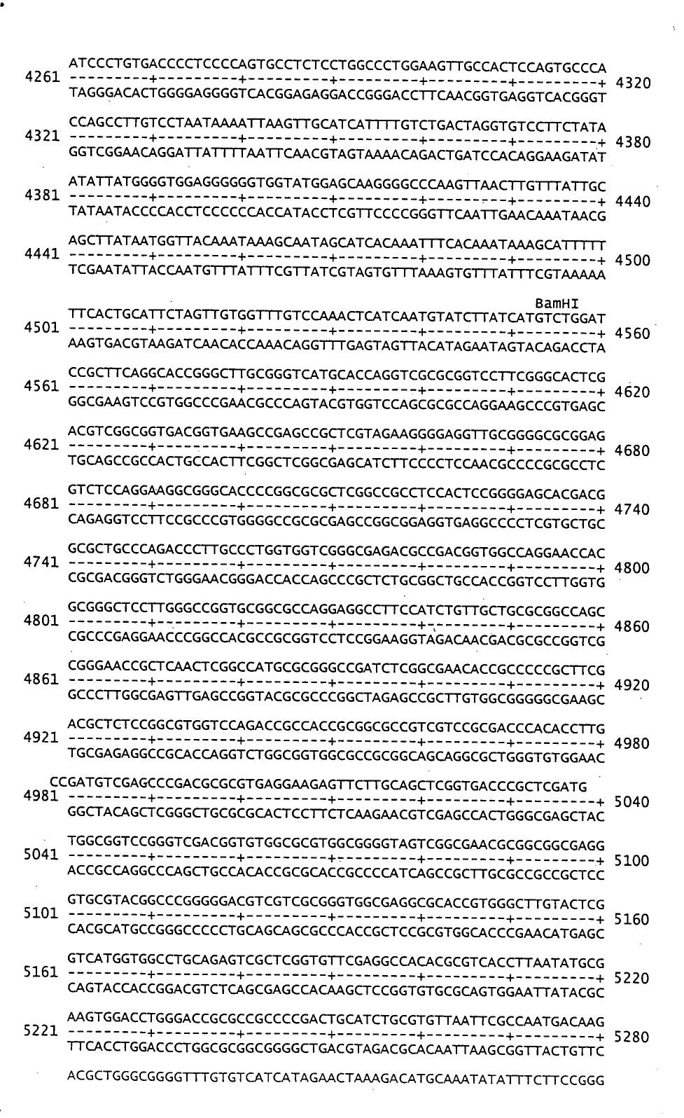

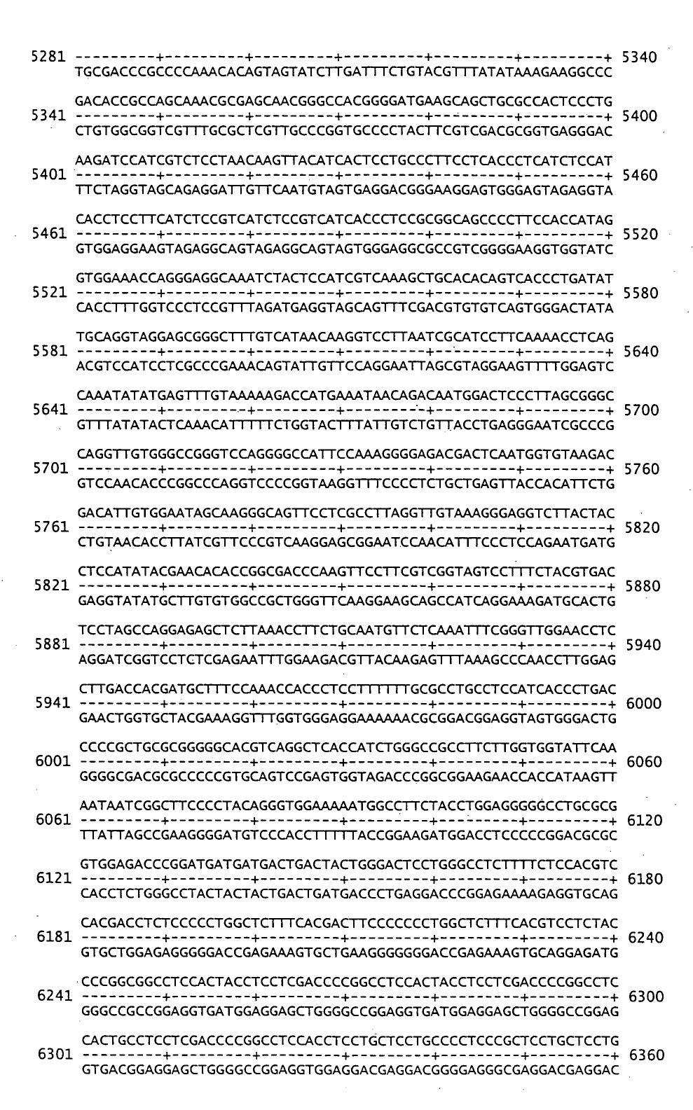

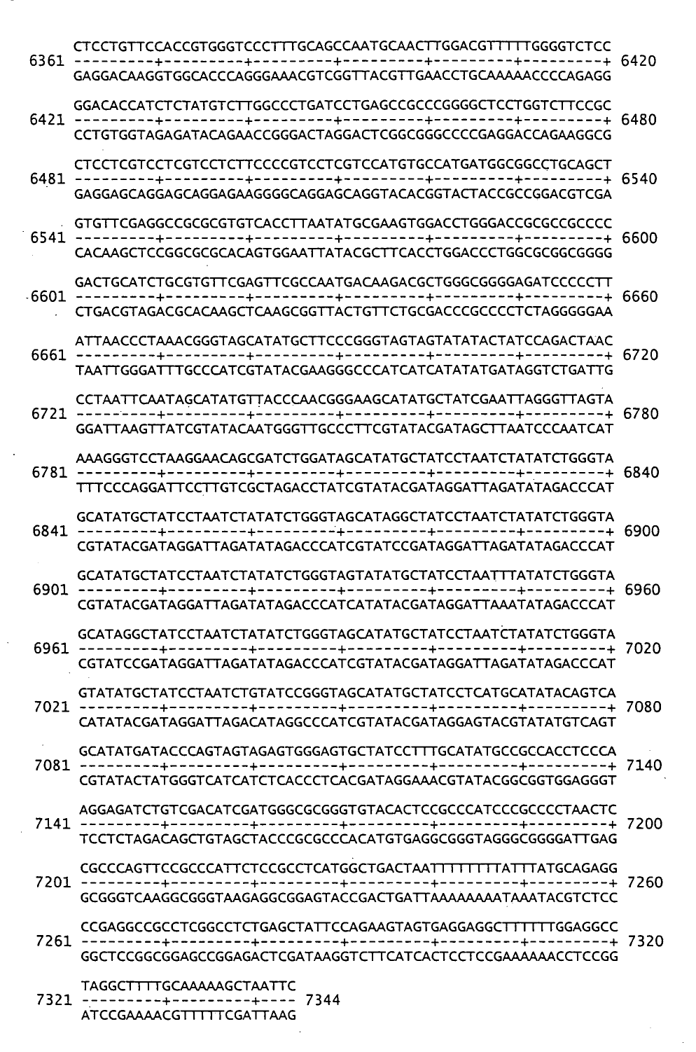

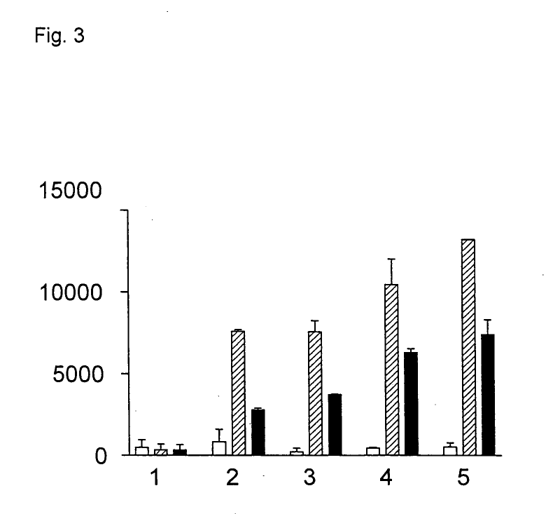

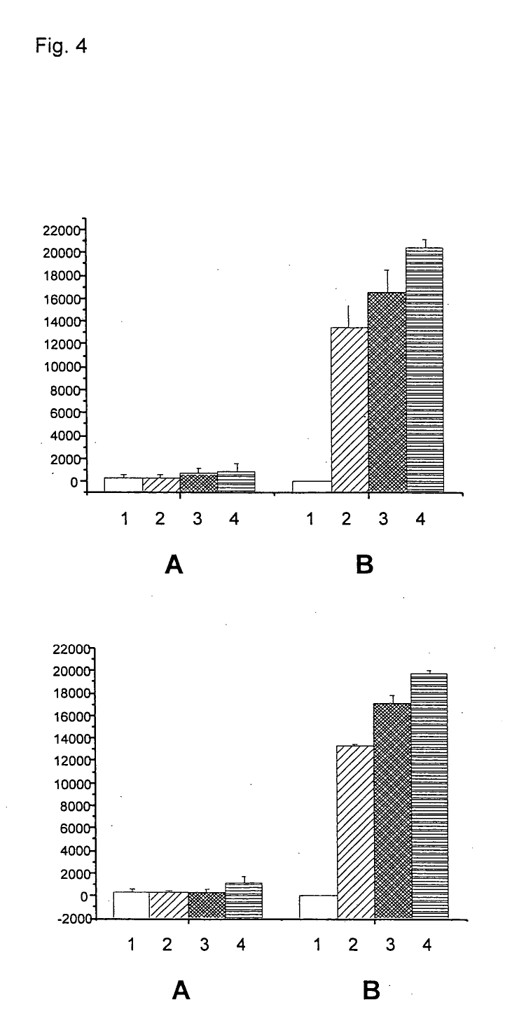

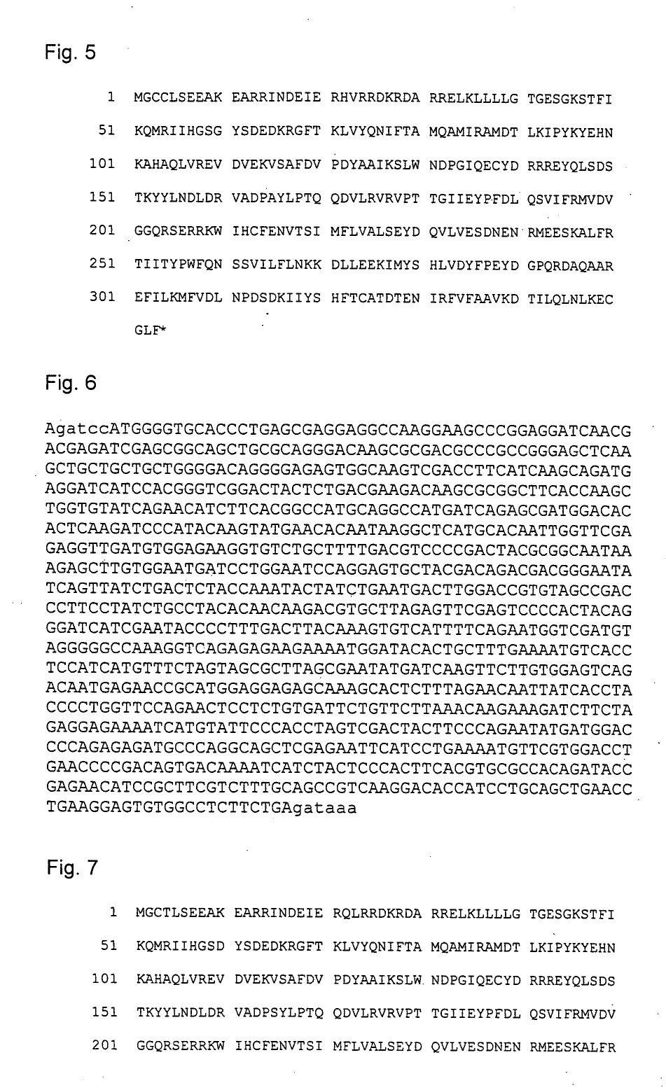

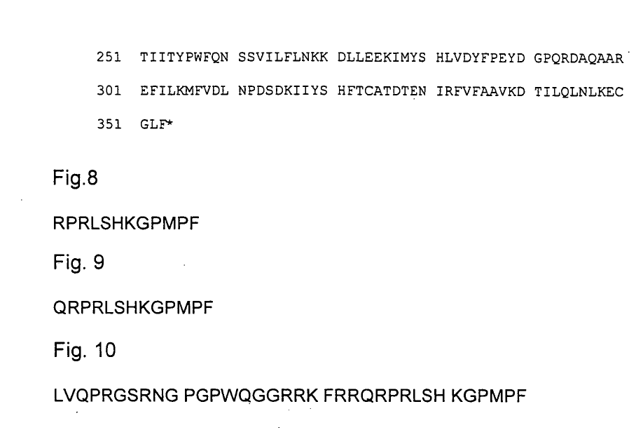

Stably transfected HEK 293 cell line (overexpressing G_{αi4qi4} gene) was grown on 100-mm plates with 10 ml DMEM (10% FCS). After 24 hours, medium was removed and 8 ml fresh DMEM (10% FCS) was added. 12 μl FuGENE 6 transfection reagent (Roche, Germany) and 400 μl OptiMEM was mixed and incubated for 5 min. Thereafter 4 μg endotoxin-free plasmid DNA (APJ/pEAK8; see FIG. 2) was added and samples were incubated for another 10 min. The transfection mixture was added to the cells. After 24 hours medium was exchanged with fresh medium containing zeocin (250 μg/ml) and puromycin (500 ng/ml). To select stable cell lines cells were cultivated for 6 weeks prior to use in functional tests. Cells without antibiotic resistance died after approximately 2 weeks.

2. FLI PR Calcium Assay

The FLIPR calcium assay provides a suitable method for detecting changes in intracellular calcium concentration. It is a fluorescence-based assay which was performed according to the manufacture's instructions (Molecular Devices, CA, USA). Briefly summarized, cells were seeded on 96- or 384-well plates Loading Buffer was added to the cells and plates incubated for 1 hour at 37° C. After incubation cells were stimulated with the prevailing APJ receptor ligand or none. Thereafter intracellular calcium concentration was measured in the FLIPR according to the FLIPR system manual.

2.1 Chemicals and Equipment

| | |
|---|---|
| Materials for cell culture | |
| DMEM | Gibco 41965-039 |
| FCS gold | PAA A15-649, Chg.: A01121-170 |
| NON-ESSENTIAL AMINO ACIDS | Gibco 11140-035 |
| (NEAs) | |
| Zeocin | Invitrogen 45-0003 |
| Puromycin × 2 HCl | MoBiTec 80018 |
| TRYPSIN/EDTA solution | Biochrom AG L2143 |
| PBS w/o Ca and Mg | GIBCO 14190-094 |
| Materials for FLIPR assay | |
| Probenicid | Sigma P-8761 |
| Pluronic F127 | Molecular probes P-3000 |
| Fluo-4/AM | Molecular probes F-14202 |
| Apelin 12 | Phoenix 057-23 |
| Apelin 13 | Bachem H-4566 |
| Apelin 36 | Bachem H-4896 |
| Pyr-Apelin 13 | Bachem H-4568 |

| | | |
|---|---|---|
| Cell culture medium | 500 ml DMEM | Final concentration |
| | 50 ml FCS | 10% |
| | 5.5 ml NEAs | 1× |
| | 1.389 ml Zeocin | 250 μg/ml |
| | 0.278 ml Puromycin | 500 ng/ml |
| Assay and dilution buffer | 1.5 ml 1 M CaCl₂ | 1.5 mM |
| | 27.0 ml 5 M NaCl | 135 mM |
| | 5.0 ml 1 M KCl | 5 mM |
| | 1.0 ml 1 M MgSO4 | 1 mM |
| | 20.0 ml 1 M HEPES | 20 mM |
| | pH7.4 | |
| | 5.0 ml 1 M Glucose | 5 mM |
| | 10.0 ml 250 mM | 2.5 mM |
| | Probenicid | |
| | in 1 N NaOH | |
| | ad 1 l, pH7.4 with HCl | |
| | conc. | |
| Dye loading buffer | Mix: | |
| | 24 μl Pluronic F127 + | 0.046% |
| | 24 μl Fluo-4, AM | |
| | Add to | |
| | 10.5 ml assay buffer | |
| Peptide solutions | All peptides were solved | Molecular |
| (store at +4° C.) | in water ad | weight/peptide |
| | | content |
| Apelin 12 | 140.57 μM | 1422.73 |
| Apelin 13 | 493.27 μM | 1550.86 g/mol/ |
| | | 76.5% |
| Apelin 36 | 173.5 μM | 4195.9 g/mol/ |
| | | 72.8% |
| Pyr-Apelin 13 | 466.6 μM | 1534.8 g/mol/ |
| | | 71.6% |

| | | |
|---|---|---|
| Equipment | | |
| Tissue Culture Flasks, | Greiner | 658 175 |
| 75 cm² | | |
| Filter cap | | |
| 96 Well Black/Clear Plate | Becton Dickinson | 35 4640 |
| Poly-D-Lysine Cellware | | |
| PS Microplate 96 well, flat | Greiner | 655101 |
| bottom | | |
| PS Cover plate | Greiner | 656101 |
| Multi channel pipette (8 | Micronic | |
| well, 12 well) | | |
| Z2-Coulter counter | Beckman | 9914558 |
| Cell washer Columbus | Tecan | F 109204 |
| plus | | |
| FLIPR | Molecular Dynamics | |

2.2 Cell Line: HEK293-PSCi4qi4-APJ

HEK293-PSCi4qi4 cells were stably transfected with pEAK8-APJ. Cells are grown in DMEM medium with 10% FCS supplemented with zeocin (250 μg/ml) and puromycin (500 ng/ml).

2.3 FLIPR Assay Procedure (Established for 96-Well Format)

For measuring calcium transients cells are grown in flat-bottom, black-wall 96-well poly-D-lysine plates. Cells are seeded at a density of 60,000 cells/well in 200 μl and cultured over night (ca. 18 h).

For dye-loading the culture medium is carefully removed and replaced by 100 μl/well dye loading buffer. Cells are incubated for 1 h at 37° C., and then washed 3× with assay buffer. After washing 90 μl buffer/well are left.

Apelin peptides were added in 90 μl assay buffer (2× concentrated).

Parameters of measurement: Sample interval: 2 sec
Sample count: 60
Fluid volume: 90 μl

After sample: 6
Pipette height: 130 μl
Dispense speed: 45 μl/sec
Mix after addition: yes
   2.4 Determination of EC50 of Apelin Peptides

EC50 values of apelin peptides were determined in two independent measurements:

| EC50 | nM | nM |
|---|---|---|
| Apelin-12 | 57 | 61 |
| Apelin-13 | 29 | 37 |
| Apelin-36 | 51 | 27 |
| Pyr-apelin-13 | 34 | 34 |

2.5 Determination of Z′ Factor

The Z′ factor is defined by

$${Z}^{′} = 1 - \frac{3 * SD high control + 3 * SD low control}{mean high control - mean low control}$$

and is used as a measure to judge the suitability of an assay for high throughput screening. Interpretation of the Z′ factor is aided by a classification scheme as shown.

Z′ factor was determined in two independent measurements using 500 nM apelin-13 in one half and assay buffer in the other half of a 96-well plate. Calculated mean Z′ factor was 0.91.

| | |
|---|---|
| (Classification: | |
| ideal assay | z = 1 |
| excellent assay | 1 > z ≧ 0.5 |
| ± acceptable | 0.5 > z > 0 |
| ‘yes/no’ type | z = 0 |
| screening impossible | z < 0) |

2.6 Determination of DMSO Dependency

EC50 values of apelin-13 were determined in presence of increasing concentrations of DMSO (0-10%). A maximum final concentration of 2% DMSO in the assay is tolerated without influencing the EC50 value of apelin-13.

2.7 Determination of Stability of Apelin Peptides

According to the manufacturer's instructions apelin peptides were solved in sterile water and stored at +4° C. Thus, apelin peptides are stable and give reproducible EC50 values. When left at room temperature for 5 hours (in the final concentration in assay buffer), EC50 values of apelin peptides drop by a factor of at least 2-fold indicating that peptides are not stable under these conditions.

3. Gene Expression Levels of APJ Receptor and Apelin in Normal and Diseased Heart

A gene expression library providing expression profiles for a total number of 514 well defined normal and diseased heart samples, accompanied by clinical patient information was generated according to standard methods known to the skilled in the art. Gene expression in normal and diseased heart was compared using HG-U95A-E GeneChips (Affymetrix, Santa Clara, Calif.). The APJ receptor gene was upregulated in diseased heart more than sixfold (Table 1). More detailed analysis indicated that APJ receptor is significantly upregulated in heart samples from patients with congestive dilated cardiomyopathy (n=124), coronary artery disease (n=301) and hypertrophic cardiomyopathy (n=37) when compared to normal nonfailing heart as control (n=52). A similar expression pattern could be obtained from apelin, the endogenous peptide ligand for APJ receptor, where the gene was upregulated in diseased heart more than twofold. No significant differences could be observed in the expression pattern for both genes in comparison of atrium versus ventricle or left versus right heart (data not shown).

1. A test system for the identification of an APJ receptor ligand comprising
(a) an APJ receptor or a functional variant thereof, and
(b) a Gα protein which is selected from the group consisting of G_{αΔ6qi4myr} protein, G_{αi4qi4} protein and G_{α16} protein.

2. The test system according to claim 1 further comprising a test ligand.

3. The test system according to claim 2, wherein said test ligand is an agonist.

4. The test system according to claim 3, wherein said agonist is a full, partial or inverse agonist.

5. The test system according to claim 2 wherein said test ligand is an antagonist.

6. The test system as in any of claims 1-5, wherein the APJ receptor is a mammalian APJ receptor or a variant thereof.

7. The test system according to claim 6, wherein the APJ receptor is a human, mouse or rat APJ receptor or a variant thereof.

8. The test system according to claim 7, wherein the APJ receptor is the human APJ receptor or a variant thereof.

9. The test system as in any of claims 1-4, wherein the test system is a genetically engineered cell and wherein at least one of said APJ receptor or functional variant thereof or said Gα protein is introduced into said cell.

10. The test system according to claim 9, wherein said genetically engineered cell is a transient or stable transfected cell line or membrane thereof.

11. A method of screening an APJ receptor ligand, wherein the method comprises the steps of:
(a) providing an APJ receptor or a functional variant thereof, and a Gα protein which is selected from the group consisting of G_{αΔ6qi4myr} protein, G_{αi4qi4} protein and G_{α16} protein,
(b) providing a test compound,
(c) optionally providing a known APJ receptor ligand, and
(d) measuring or detecting the influence of the test compound on the APJ receptor-mediated signal transduction pathway.

12. The method of screening according to claim 11, wherein said ligand is an agonist.

13. The method of screening according to claim 12, wherein said agonist is a full, partial or inverse agonist.

14. The method of screening according to claim 11, wherein said ligand is an antagonist.

15. The method as in any of claims 11-14, wherein said test compound is provided in the form of a chemical compound library.

16. The method as in any of claims 11-14, wherein the method is carried out using whole cells.

17. The method as in any of claims 11-14, wherein the method is carried out in a robotics system.

18. The method as in any of claims 11-14, wherein the method is a method of high-through put screening of an APJ receptor ligand.

19. A method for producing a medicament, wherein the method comprises the steps of:
(a) identifying a APJ receptor ligand carrying out the method as in any of claims 11-14,
(b) providing adequate amounts of said ligand, and
(c) formulating the ligand with one or more pharmaceutically acceptable carriers or auxiliary substances.

20. A method for diagnosis or prognosis of a heart disease comprising the following steps:
(a) determining the expression level of APJ receptor gene or apelin gene in a sample to be tested,
(b) comparing the expression level of APJ receptor or apelin in said sample with the expression level of APJ receptor or apelin in a control probe, and
(c) identifying said sample with increased APJ receptor gene expression or apelin gene expression.

21. The method according to claim 20, wherein the expression levels of the APJ receptor gene or apelin gene is determined on the basis of levels of mRNAs transcribed from the gene, or levels of polypeptides translated from the gene.

22. The method according to claim 21, wherein the levels of mRNAs are determined by a hybridization method or a nucleic acid amplification method.

23. The method according to claim 21, wherein the levels of polypeptides are determined by using binding proteins capable of binding specifically to the polypeptides or fragments thereof.


SEQUENCE LISTING (8 sequences)

SEQ ID NO: 1 - DNA, 1080 bp, Artificial
  Mouse
bamhggatccatggggtgctgcctgagcgaggaggccaaggaagcccggcggatcaacgacgagatcgagcggcacgtccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgacgtccccgactacgcggcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtgtggcctcttctaaatgcatns

SEQ ID NO: 2 - DNA, 14987 bp, Artificial
  Human
ccaccgctaccagcggtggtttgtttgccggatcaagagctaccaactctttttccgaagggtggcgatggtcgccaccaaacaaacggcctagttctcgatggttgagaaaaaggcttcgtaactggcttcagcagagcgcagataccaaatactgtccttctagtgtagccgtagttacattgaccgaagtcgtctcgcgtctatggtttatgacaggaagatcacatcggcatcaatggccaccacttcaagaactctgtagcaccgcctacatacctcgctctgctaatcctgttaccggtggtgaagttcttgagacatcgtggcggatgtatggagcgagacgattaggacaatccagtggctgctgccagtggcgataagtcgtgtcttaccgggttggactcaagacgatagggtcaccgacgacggtcaccgctattcagcacagaatggcccaacctgagttctgctatcttaccggataaggcgcagcggtcgggctgaacggggggttcgtgcacacagcccagcttgaatggcctattccgcgtcgccagcccgacttgccccccaagcacgtgtgtcgggtcgaacgagcgaacgacctacaccgaactgagatacctacagcgtgagcattgagaaagcgccacgctcgcttgctggatgtggcttgactctatggatgtcgcactcgtaactctttcgcggtgccttcccgaagggagaaaggcggacaggtatccggtaagcggcagggtcggaacaggagaggaagggcttccctctttccgcctgtccataggccattcgccgtcccagccttgtcctctccgcacgagggagcttccagggggaaacgcctggtatctttatagtcctgtcgggtttcgcgcgtgctccctcgaaggtccccctttgcggaccatagaaatatcaggacagcccaaagcgcacctctgacttgagcgtcgatttttgtgatgctcgtcaggggggcggagcctatggaaagtggagactgaactcgcagctaaaaacactacgagcagtccccccgcctcggatacctttaacgccagcaacgcaagctagagtttaaacttgacagatgagacaataaccctgataaatttgcggtcgttgcgttcgatctcaaatttgaactgtctactctgttattgggactatttagcttcaataatattgaaaaaggaaaagtatgagtattcaacatttccgtgtcgcccttatcgaagttattataactttttccttttcatactcataagttgtaaaggcacagcgggaatatcccttttttgcggcattttgccttcctgtttttgctcacccagaaacgctggtgaaagtagggaaaaaacgccgtaaaacggaaggacaaaaacgagtgggtctttgcgaccactttcaaaaagatgcagaagatcacttgggtgcgcgagtgggttacatcgaactggatctcaacagttttctacgtcttctagtgaacccacgcgctcacccaatgtagcttgacctagagttgtccggtaagatccttgagagttttcgccccgaagaacgtttcccaatgatgagcacttttaagccattctaggaactctcaaaagcggggcttcttgcaaagggttactactcgtgaaaattagttctgctatgtggcgcggtattatcccgtattcttggttgaatactcaccagtcacagtcaagacgatacaccgcgccataatagggcataagaaccaacttatgagtggtcagtgtcaaaagcatcttacggatggcatgacagtaagagaattatgcagtgctgccataaccatgattttcgtagaatgcctaccgtactgtcattctcttaatacgtcacgacggtattggtactgtgataacactgcggccaacttacttctgacaactatcggaggaccgaaggagctaaccgcactattgtgacgccggttgaatgaagactgttgatagcctcctggcttcctcgattggccttttttgcacaacatgggggatcatgtaactcgccttgatcgttgggaaccggagctgagaaaaaacgtgttgtaccccctagtacattgagcggaactagcaacccttggcctcgactatgaagccataccaaacgacgagcgtgacaccacgatgcctgtagcaatggcaacaacgttacttcggtatggtttgctgctcgcactgtggtgctacggacatcgttaccgttgttgcatgcgaaaactattaactggcgaactacttactctagcttcccggcaacaactaatagactacgcttttgataattgaccgcttgatgaatgagatcgaagggccgttgttgattatctgaggatggaggcggataaagttgcaggaccacttctgcgctcggcacttccggctggctggtcctacctccgcctatttcaacgtcctggtgaagacgcgagccgtgaaggccgaccgaccattattgctgataaatcaggagccggtgagcgtgggtcacgcggtatcattgcagcactggaataacgactatttagtcctcggccactcgcacccagtgcgccatagtaacgtcgtgaccggccggatggtaagccctcccgtatcgtagttatctacactacggggagtcaggcaactaccggcctaccattcgggagggcatagcatcaatagatgtgatgcccctcagtccgttgattggatgaacgaaatagacagatcgctgagataggtgcctcactgattaagcattggtaagacctacttgctttatctgtctagcgactctatccacggagtgactaattcgtaaccattcgataaatttctggtaaggaggacacgtatggaagtgggcaagttggggaagccgtatccgctatttaaagaccattcctcctgtgcataccttcacccgttcaaccccttcggcataggcttgctgaatctggcatatgtgggagtataagacgcgcagcgtcgcatcaggcatttttttaacgacttagaccgtatacaccctcatattctgcgcgtcgcagcgtagtccgtaaaaaaactgcgccaatgcaaaaaggccatccgtcaggatggcctttcgcataactagtgaggctccgacgcggttacgtttttccggtaggcagtcctaccggaaagcgtattgatcactccgaggggtgcccgtcagtgggcagagcgcacatcgcccacagtccccgagaagttggggggagggccacgggcagtcacccgtctcgcgtgtagcgggtgtcaggggctcttcaacccccctcccgtcggcaattgaaccggtgcctagagaaggtggcgcggggtaaactgggaaagtgatgtccagccgttaacttggccacggatctcttccaccgcgccccatttgaccctttcactacaggtgtactggctccgcctttttcccgagggtgggggagaaccgtatataagtgcagtagtccacatgaccgaggcggaaaaagggctcccaccccctcttggcatatattcacgtcatcaggccgtgaacgttctttttcgcaacgggtttgccgccagaacacaggtaagtgccgtgtgtcggcacttgcaagaaaaagcgttgcccaaacggcggtcttgtgtccattcacggcacacaggttcccgcgggcctggcctctttacgggttatggcccttgcgtgccttgaattacttccccaagggcgcccggaccggagaaatgcccaataccgggaacgcacggaacttaatgaaggacctggctgcagtacgtgattcttgatcccgagcttcgggttggaagtgggtgggagagttggaccgacgtcatgcactaagaactagggctcgaagcccaaccttcacccaccctctcatcgaggccttgcgcttaaggagccccttcgcctcgtgcttgagttgaggcctggcctgggagctccggaacgcgaattcctcggggaagcggagcacgaactcaactccggaccggaccccgctggggccgccgcgtgcgaatctggtggcaccttcgcgcctgtctcgctgctttcgatgcgaccccggcggcgcacgcttagaccaccgtggaagcgcggacagagcgacgaaagctaaagtctctagccatttaaaatttttgatgacctgctgcgacgctttttttctggcaagatttcagagatcggtaaattttaaaaactactggacgacgctgcgaaaaaaagaccgttctaagtcttgtaaatgcgggccaagatcgatctgcacactggtatttcggtttttggggccgctcagaacatttacgcccggttctagctagacgtgtgaccataaagccaaaaaccccggcggggcggcgacggggcccgtgcgtcccagcgcacatgttcggcgaggcggggcctgcgagccccgccgctgccccgggcacgcagggtcgcgtgtacaagccgctccgccccggacgctcggcggccaccgagaatcggacgggggtagtctcaagctggccggcctgctctggtgcctggcgccggtggctcttagcctgcccccatcagagttcgaccggccggacgagaccacggacccctcgcgccgccgtgtatcgccccgccctgggcggcaaggctggcccggtcggcaccagtggagcgcggcggcacatagcggggcgggacccgccgttccgaccgggccagccgtggtcatgcgtgagcggaaagatggccgcttcccggccctgctgcagggagctcaaaatggaggacacgcactcgcctttctaccggcgaagggccgggacgacgtccctcgagttttacctcctggcggcgctcgggagagcgggcgggtgagtcacccacacaaaggaaaagggcctttccgtccgccgcgagccctctcgcccgcccactcagtgggtgtgtttccttttcccggaaaggcagctcagccgtcgcttcatgtgactccacggagtaccgggcgccgtccaggcacctcgattagagtcggcagcgaagtacactgaggtgcctcatggcccgcggcaggtccgtggagctaathgttctcgagcttttggagtacgtcgtctttaggttggggggaggggttttatgcgatggacaagagctcgaaaacctcatgcagcagaaatccaacccccctccccaaaatacgctacctdragtttccccacactgagtgggtggagactgaagttaggccagcttggcacttgatgtaattcaaaggggtgtgactcacccacctctgacttcaatccggtcgaaccgtgaactacattaactccttggaatttgccctttttgagtttggatcttggttcattctcaagcctcagacagtgaggaaccttaaacgggaaaaactcaaacctagaaccaagtaagagttcggagtctgtcahndggttcaaagtttttttcttccatttcaggtgtcgtgaaaagcttgccaccatggactacaccaagtttcaaaaaaagaaggtaaagtccacagcacttttcgaacggtggtacctgatgtrcntycraggacgacgacgacaagaacatgtggggcgaattaattccttaagctatcaacaagtttgtcctgctgctgctgttcttgtacaccccgcttaattaaggaattcgatagttgttcaaactacaaaaaagcaggcttaggaatggaggaaggtggtgattttgacaactactatggggcaatgttttttcgtccgaatccttacctccttccaccactaaaactgttgatgataccccgtmggddnyygagacaaccagtctgagtgtgagtacacagactggaaatcctcgggggccctcatccctgccctgttggtcagactcacactcatgtgtctgacctttaggagcccccgggagtagggacggdnscytdwkssgaaatctacatgttggtcttcctcctgggcaccacgggaaacggtctggtgctctggaccgtgtagatgtacaaccagaaggaggacccgtggtgccctttgccagaccacgagacctggcacymvgttgngvwtvcrvtttcggagcagccgggagaagaggcgctcagctgatatcttcattgctagcctggcggtgaaagcctcgtcggccctcttctccgcgagtcgactatagaagtaacgatcggaccgccacrssrkrrsadasavgctgacctgaccttcgtggtgacgctgcccctgtgggctacctacacgtaccgggactatcgactggactggaagcaccactgcgacggggacacccgatggatgtgcatggccctgataadtvvtwatytyrdygactggccctttgggaccttcttctgcaagctcagcagctacctcatcttcgtcaacatgctgaccgggaaaccctggaagaagacgttcgagtcgtcgatggagtagaagcagttgtacdwgtckssyvnmtacgccagcgtcttctgcctcaccggcctcagcttcgaccgctacctggccatcgtgaggatgcggtcgcagaagacggagtggccggagtcgaagctggcgatggaccggtagcactccyasvctgsdryavrccagtggccaatgctcggctgaggctgcgggtcagcggggccgtggccacggcagttcttggtcaccggttacgagccgactccgacgcccagtcgccccggcaccggtgccgtcaagaavanarrrvsgavatavtgggtgctggccgccctcctggccatgcctgtcatggtgttacgcaccaccggggacttgacccacgaccggcgggaggaccggtacggacagtaccacaatgcgtggtggcccctgaacwvaaamvmvrttgdgagaacaccactaaggtgcagtgctacatggactactccatggtggccactgtgagctcactcttgtggtgattccacgtcacgatgtacctgatgaggtaccaccggtgacactcgagtnttkvcymdysmvatvssgagtgggcctgggaggtgggccttggggtctcgtccaccaccgtgggctttgtggtgcccctcacccggaccctccacccggaaccccagagcaggtggtggcacccgaaacaccacgggwawvggvssttvgvvttcaccatcatgctgacctgttacttcttcatcgcccaaaccatcgctggccacttccgcaagtggtagtacgactggacaatgaagaagtagcgggtttggtagcgaccggtgaaggcgtmtcyataghraaggaacgcatcgagggcctgcggaagcggcgccggctgctcagcatcatcgtggtgctgttccttgcgtagctcccggacgccttcgccgcggccgacgagtcgtagtagcaccacgackrgrkrrrsvvdragtggtgacctttgccctgtgctggatgccctaccacctggtgaagacgctgtacatgctgcaccactggaaacgggacacgacctacgggatggtggaccacttctgcgacatgtacgacvvtacwmyhvktymggcagcctgctgcactggccctgtgactttgacctcttcctcatgaacatcttcccctacccgtcggacgacgtgaccgggacactgaaactggagaaggagtacttgtagaaggggatggshwcddmnytgcacctgcatcagctacgtcaacagctgcctcaaccccttcctctatgcctttttcgacacgtggacgtagtcgatgcagttgtcgacggagttggggaaggagatacggaaaaagctgctcsyvnscnyadccccgcttccgccaggcctgcacctccatgctctgctgtggccagagcaggtgcgcaggcggggcgaaggcggtccggacgtggaggtacgagacgacaccggtctcgtccacgcgtccgrractsmccgsrcagacctcccacagcagcagtggggagaagtcagccagctactcttcggggcacagccaggggtggagggtgtcgtcgtcacccctcttcagtcggtcgatgagaagccccgtgtcggtcccctshsssgksasyssghsgcccggccccaacatgggcaagggtggagaacagatgcacgagaaatccatcccctacagcgggccggggttgtacccgttcccacctcttgtctacgtgctctttaggtaggggatgtcggnmgkggmhksyscaggagacccttgtggttgactaggacccagctttcttgtacaaagtggttgatagcttggtcctctgggaacaccaactgatcctgggtcgaaagaacatgtttcaccaactatcgaactvvdbamhrcntycrgtaccgagctcggatccactagtccagtgtggtggaattggccgcaggtaagccagcccacatggctcgagcctaggtgatcaggtcacaccaccttaaccggcgtccattcggtcgggtggcctcgccctccagctcaaggcgggacaggtgccctagagtagcctgcatccagggacaccggagcgggaggtcgagttccgccctgtccacgggatctcatcggacgtaggtccctgtggccccagccgggtgctgacacgtccacctccatctcttcctcaggtctgcccgggtggcccggggtcggcccacgactgtgcaggtggaggtagagaaggagtccagacgggcccaccgatccctgtgacccctccccagtgcctctcctggccctggaagttgccactccagtgcccatagggacactggggaggggtcacggagaggaccgggaccttcaacggtgaggtcacgggtccagccttgtcctaataaaattaagttgcatcattttgtctgactaggtgtccttctataggtcggaacaggattattttaattcaacgtagtaaaacagactgatccacaggaagatatatattatggggtggaggggggtggtatggagcaaggggcccaagttaacttgtttattgctataataccccacctccccccaccatacctcgttccccgggttcaattgaacaaataacgagcttataatggttacaaataaagcaatagcatcacaaatttcacaaataaagcattttttcgaatattaccaatgtttatttcgttatcgtagtgtttaaagtgtttatttcgtaaaaabamhttcactgcattctagttgtggtttgtccaaactcatcaatgtatcttatcatgtctggataagtgacgtaagatcaacaccaaacaggtttgagtagttacatagaatagtacagacctaccgcttcaggcaccgggcttgcgggtcatgcaccaggtcgcgcggtccttcgggcactcgggcgaagtccgtggcccgaacgcccagtacgtggtccagcgcgccaggaagcccgtgagcacgtcggcggtgacggtgaagccgagccgctcgtagaaggggaggttgcggggcgcggagtgcagccgccactgccacttcggctcggcgagcatcttcccctccaacgccccgcgcctcgtctccaggaaggcgggcaccccggcgcgctcggccgcctccactccggggagcacgacgcagaggtccttccgcccgtggggccgcgcgagccggcggaggtgaggcccctcgtgctgcgcgctgcccagacccttgccctggtggtcgggcgagacgccgacggtggccaggaaccaccgcgacgggtctgggaacgggaccaccagcccgctctgcggctgccaccggtccttggtggcgggctccttgggccggtgcggcgccaggaggccttccatctgttgctgcgcggccagccgcccgaggaacccggccacgccgcggtcctccggaaggtagacaacgacgcgccggtcgcgggaaccgctcaactcggccatgcgcgggccgatctcggcgaacaccgcccccgcttcggcccttggcgagttgagccggtacgcgcccggctagagccgcttgtggcgggggcgaagcacgctctccggcgtggtccagaccgccaccgcggcgccgtcgtccgcgacccacaccttgtgcgagaggccgcaccaggtctggcggtggcgccgcggcagcaggcgctgggtgtggaacccgatgtcgagcccgacgcgcgtgaggaagagttcttgcagctcggtgacccgctcgatgggctacagctcgggctgcgcgcactccttctcaagaacgtcgagccactgggcgagctactggcggtccgggtcgacggtgtggcgcgtggcggggtagtcggcgaacgcggcggcgaggaccgccaggcccagctgccacaccgcgcaccgccccatcagccgcttgcgccgccgctccgtgcgtacggcccgggggacgtcgtcgcgggtggcgaggcgcaccgtgggcttgtactcgcacgcatgccgggccccctgcagcagcgcccaccgctccgcgtggcacccgaacatgagcgtcatggtggcctgcagagtcgctcggtgttcgaggccacacgcgtcaccttaatatgcgcagtaccaccggacgtctcagcgagccacaagctccggtgtgcgcagtggaattatacgcaagtggacctgggaccgcgccgccccgactgcatctgcgtgttaattcgccaatgacaagttcacctggaccctggcgcggcggggctgacgtagacgcacaattaagcggttactgttcacgctgggcggggtttgtgtcatcatagaactaaagacatgcaaatatatttcttccgggtgcgacccgccccaaacacagtagtatcttgatttctgtacgtttatataaagaaggcccgacaccgccagcaaacgcgagcaacgggccacggggatgaagcagctgcgccactccctgctgtggcggtcgtttgcgctcgttgcccggtgcccctacttcgtcgacgcggtgagggacaagatccatcgtctcctaacaagttacatcactcctgcccttcctcaccctcatctccatttctaggtagcagaggattgttcaatgtagtgaggacgggaaggagtgggagtagaggtacacctccttcatctccgtcatctccgtcatcaccctccgcggcagccccttccaccataggtggaggaagtagaggcagtagaggcagtagtgggaggcgccgtcggggaaggtggtatcgtggaaaccagggaggcaaatctactccatcgtcaaagctgcacacagtcaccctgatatcacctttggtccctccgtttagatgaggtagcagtttcgacgtgtgtcagtgggactatatgcaggtaggagcgggctttgtcataacaaggtccttaatcgcatccttcaaaacctcagacgtccatcctcgcccgaaacagtattgttccaggaattagcgtaggaagttttggagtccaaatatatgagtttgtaaaaagaccatgaaataacagacaatggactcccttagcgggcgtttatatactcaaacatttttctggtactttattgtctgttacctgagggaatcgcccgcaggttgtgggccgggtccaggggccattccaaaggggagacgactcaatggtgtaagacgtccaacacccggcccaggtccccggtaaggtttcccctctgctgagttaccacattctggacattgtggaatagcaagggcagttcctcgccttaggttgtaaagggaggtcttactacctgtaacaccttatcgttcccgtcaaggagcggaatccaacatttccctccagaatgatgctccatatacgaacacaccggcgacccaagttccttcgtcggtagtcctttctacgtgacgaggtatatgcttgtgtggccgctgggttcaaggaagcagccatcaggaaagatgcactgtcctagccaggagagctcttaaaccttctgcaatgttctcaaatttcgggttggaacctcaggatcggtcctctcgagaatttggaagacgttacaagagtttaaagcccaaccttggagcttgaccacgatgctttccaaaccaccctccttttttgcgcctgcctccatcaccctgacgaactggtgctacgaaaggtttggtgggaggaaaaaacgcggacggaggtagtgggactgccccgctgcgcgggggcacgtcaggctcaccatctgggccgccttcttggtggtattcaaggggcgacgcgcccccgtgcagtccgagtggtagacccggcggaagaaccaccataagttaataatcggcttcccctacagggtggaaaaatggccttctacctggagggggcctgcgcgttattagccgaaggggatgtcccacctttttaccggaagatggacctcccccggacgcgcgtggagacccggatgatgatgactgactactgggactcctgggcctcttttctccacgtccacctctgggcctactactactgactgatgaccctgaggacccggagaaaagaggtgcagcacgacctctccccctggctctttcacgacttccccccctggctctttcacgtcctctacgtgctggagagggggaccgagaaagtgctgaagggggggaccgagaaagtgcaggagatgcccggcggcctccactacctcctcgaccccggcctccactacctcctcgaccccggcctcgggccgccggaggtgatggaggagctggggccggaggtgatggaggagctggggccggagcactgcctcctcgaccccggcctccacctcctgctcctgcccctcccgctcctgctcctggtgacggaggagctggggccggaggtggaggacgaggacggggagggcgaggacgaggacctcctgttccaccgtgggtccctttgcagccaatgcaacttggacgtttttggggtctccgaggacaaggtggcacccagggaaacgtcggttacgttgaacctgcaaaaaccccagaggggacaccatctctatgtcttggccctgatcctgagccgcccggggctcctggtcttccgccctgtggtagagatacagaaccgggactaggactcggcgggccccgaggaccagaaggcgctcctcgtcctcgtcctcttccccgtcctcgtccatgtgccatgatggcggcctgcagctgaggagcaggagcaggagaaggggcaggagcaggtacacggtactaccgccggacgtcgagtgttcgaggccgcgcgtgtcaccttaatatgcgaagtggacctgggaccgcgccgcccccacaagctccggcgcgcacagtggaattatacgcttcacctggaccctggcgcggcgggggactgcatctgcgtgttcgagttcgccaatgacaagacgctgggcggggagatcccccttctgacgtagacgcacaagctcaagcggttactgttctgcgacccgcccctctagggggaaattaaccctaaacgggtagcatatgcttcccgggtagtagtatatactatccagactaactaattgggatttgcccatcgtatacgaagggcccatcatcatatatgataggtctgattgcctaattcaatagcatatgttacccaacgggaagcatatgctatcgaattagggttagtaggattaagttatcgtatacaatgggttgcccttcgtatacgatagcttaatcccaatcataaagggtcctaaggaacagcgatctggatagcatatgctatcctaatctatatctgggtatttcccaggattccttgtcgctagacctatcgtatacgataggattagatatagacccatgcatatgctatcctaatctatatctgggtagcataggctatcctaatctatatctgggtacgtatacgataggattagatatagacccatcgtatccgataggattagatatagacccatgcatatgctatcctaatctatatctgggtagtatatgctatcctaatttatatctgggtacgtatacgataggattagatatagacccatcatatacgataggattaaatatagacccatgcataggctatcctaatctatatctgggtagcatatgctatcctaatctatatctgggtacgtatccgataggattagatatagacccatcgtatacgataggattagatatagacccatgtatatgctatcctaatctgtatccgggtagcatatgctatcctcatgcatatacagtcacatatacgataggattagacataggcccatcgtatacgataggagtacgtatatgtcagtgcatatgatacccagtagtagagtgggagtgctatcctttgcatatgccgccacctcccacgtatactatgggtcatcatctcaccctcacgataggaaacgtatacggcggtggagggtaggagatctgtcgacatcgatgggcgcgggtgtacactccgcccatcccgcccctaactctcctctagacagctgtagctacccgcgcccacatgtgaggcgggtagggcggggattgagcgcccagttccgcccattctccgcctcatggctgactaattttttttatttatgcagagggcgggtcaaggcgggtaagaggcggagtaccgactgattaaaaaaaataaatacgtctccccgaggccgcctcggcctctgagctattccagaagtagtgaggaggcttttttggaggccggctccggcggagccggagactcgataaggtcttcatcactcctccgaaaaaacctccggtaggcttttgcaaaaagctaattcatccgaaaacgtttttcgattaag

SEQ ID NO: 3 - protein, 353 aa, Mus musculus
MGCCLSEEAKEARRINDEIERHVRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFDVPDYAAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKECGLF

SEQ ID NO: 4 - DNA, 1074 bp, Mus musculus
agatccatggggtgcaccctgagcgaggaggccaaggaagcccggaggatcaacgacgagatcgagcggcagctgcgcagggacaagcgcgacgcccgccgggagctcaagctgctgctgctggggacaggggagagtggcaagtcgaccttcatcaagcagatgaggatcatccacgggtcggactactctgacgaagacaagcgcggcttcaccaagctggtgtatcagaacatcttcacggccatgcaggccatgatcagagcgatggacacactcaagatcccatacaagtatgaacacaataaggctcatgcacaattggttcgagaggttgatgtggagaaggtgtctgcttttgacgtccccgactacgcggcaataaagagcttgtggaatgatcctggaatccaggagtgctacgacagacgacgggaatatcagttatctgactctaccaaatactatctgaatgacttggaccgtgtagccgacccttcctatctgcctacacaacaagacgtgcttagagttcgagtccccactacagggatcatcgaatacccctttgacttacaaagtgtcattttcagaatggtcgatgtagggggccaaaggtcagagagaagaaaatggatacactgctttgaaaatgtcacctccatcatgtttctagtagcgcttagcgaatatgatcaagttcttgtggagtcagacaatgagaaccgcatggaggagagcaaagcactctttagaacaattatcacctacccctggttccagaactcctctgtgattctgttcttaaacaagaaagatcttctagaggagaaaatcatgtattcccacctagtcgactacttcccagaatatgatggaccccagagagatgcccaggcagctcgagaattcatcctgaaaatgttcgtggacctgaaccccgacagtgacaaaatcatctactcccacttcacgtgcgccacagataccgagaacatccgcttcgtctttgcagccgtcaaggacaccatcctgcagctgaacctgaaggagtgtggcctcttctgagataaa

SEQ ID NO: 5 - protein, 353 aa, Mus musculus
MGCTLSEEAKEARRINDEIERQLRRDKRDARRELKLLLLGTGESGKSTFIKQMRIIHGSDYSDEDKRGFTKLVYQNIFTAMQAMIRAMDTLKIPYKYEHNKAHAQLVREVDVEKVSAFDVPDYAAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKECGLF

SEQ ID NO: 6 - protein, 12 aa, Homo sapiens
RPRLSHKGPMPF

SEQ ID NO: 7 - protein, 13 aa, Homo sapiens
QRPRLSHKGPMPF

SEQ ID NO: 8 - protein, 36 aa, Homo sapiens
LVQPRGSRNGPGPWQGGRRKFRRQRPRLSHKGPMPF